(19) United States
(12) Patent Application Publication
(10) Pub. No.: US 2015/0224209 A1
(43) Pub. Date: Aug. 13, 2015

(54) LENTIVIRAL VECTOR FOR STEM CELL GENE THERAPY OF SICKLE CELL DISEASE

(71) Applicants: Donald B. KOHN, Tarzana, CA (US); Fabrizia URBINATI, Los Angeles, CA (US); Zulema R. GARCIA, Los Angeles, CA (US); Roger HOLLIS, Sherman Oaks, CA (US); Sabine GEIGER-SCHREDELSEKER, Munchen (DE); Aaron COOPER, Los Angeles, CA (US); Shantha SENADHEERA, Castaic, CA (US)

(72) Inventors: Donald B. Kohn, Tarzana, CA (US); Fabrizia Urbinati, Los Angeles, CA (US); Zulema R. Garcia, Los Angeles, CA (US); Roger Hollis, Sherman Oaks, CA (US); Sabine Geiger-Schredelseker, Munchen (DE); Aaron Cooper, Los Angeles, CA (US); Shantha Senadheera, Castaic, CA (US)

(73) Assignee: THE REGENTS OF THE UNIVERSITY OF CALIFORNIA, Oakland, CA (US)

(21) Appl. No.: 14/427,965

(22) PCT Filed: Sep. 10, 2013
(86) PCT No.: PCT/US2013/059073

§ 371 Date: Mar. 12, 2015

Related U.S. Application Data
(60) Provisional application No. 61/701,318, filed on Sep. 14, 2012.

(51) Int. Cl. A61K 48/00; C07K 14/805; C12N 15/86

(52) U.S. Cl. CPC A61K 48/0066; C12N 15/86; C07K 14/805; C12N 2740/16043; C12N 2830/40; C12N 2830/48

(57) ABSTRACT

In various embodiments a recombinant lentiviral vector is provided comprising an expression cassette comprising a nucleic acid construct comprising an anti-sickling human beta globin gene encoding an anti-sickling-beta globin polypeptide comprising the mutations Gly16Asp, Glu22Ala and Thr87Gln, where the lentiviral vector is a TAT-independent and self-inactivating (SIN). In certain embodiments the vector additionally contains one or more insulator elements. The vectors are useful in gene therapy for the treatment of sickle cell disease.

### CROSS-REFERENCE TO RELATED APPLICATIONS

This application claims benefit of and priority to U.S. Ser. No. 61/701,318, filed on Sep. 14, 2012, which is incorporated herein by reference in its entirety for all purposes.

### STATEMENT OF GOVERNMENTAL SUPPORT

[Not Applicable]

### BACKGROUND

Sickle cell disease (SCD) is one of the most common monogenic disorders worldwide and is a major cause of morbidity and early mortality (Hoffman et al. (2009) Hematology: Basic Principles and Practice. 5th ed. London, United Kingdom, Churchill Livingstone). SCD affects approximately 80,000 Americans, and causes significant neurologic, pulmonary, and renal injury, as well as severe acute and chronic pain that adversely impacts quality of life. It is estimated that approximately 240,000 children are born annually in Africa with SCD and 80% die by their second birthday. The average lifespan of subjects with SCD in the United States is approximately 40 years and this has remained unchanged over the last 3-4 decades.

SCD is caused by a single amino acid change in β-globin (Glu 6 to Val 6) which leads to hemoglobin polymerization and red blood cell (rbc) sickling. SCD typically results in continual low-grade ischemia and episodic exacerbations or “crises” resulting in tissue ischemia, organ damage, and premature death.

Although SCD is well characterized, there is still no ideal long-term treatment. Current therapies are based on induction of fetal hemoglobin (HbF) to inhibit polymerization of sickle hemoglobin (HbS) (Voskaridou et al. (2010) Blood, 115(12): 2354-2363) and cell dehydration (Eaton and Hofrichter (1987) Blood, 70(5): 1245-1266) or reduction of the percentage of HbS by transfusions (Stamatoyannopoulos et al., eds. (2001) Molecular Basis of Blood Diseases. 3rd ed. Philadelphia, Pa., USA: WB Saunders). Allogeneic human stem cell transplantation (HSCT) from bone marrow (BM) or umbilical cord blood (UCB) or mobilized peripheral blood stem cells (mPBSC) is a potentially curative therapy, although only a small percentage of patients have undergone this procedure, mostly children with severe symptoms who had HLA-matched sibling donors (Bolaños-Meade and Brodsky (2009) Curr. Opin. Oncol. 21(2): 158-161; Rees et al. (2010) Lancet, 376(9757): 2018-2031; Shenoy (2011) Hematology Am Soc Hematol Educ Program. 2011: 273-279).

Transplantation of allogeneic cells carries the risk of graft-versus host disease (GvHD), which can be a cause of extensive morbidity. HSCT using UCB from matched unrelated donors holds reduced risk of acute or chronic GvHD compared with using BM; however, there is a higher probability of engraftment failure using UCB as a result of its lower cell dose and immunologic immaturity (Kamani et al. (2012) Biol. Blood Marrow Transplant. 18(8): 1265-1272; Locatelli and Pagliara (2012) Pediatr. Blood Cancer. 59(2): 372-376).

Gene therapy with autologous human stem cells (HSCs) is an alternative to allogeneic HSCT, since it avoids the limitations of finding a matched donor and the risks of GvHD and graft rejection. For gene therapy application in SCD patients, the safest source for autologous HSC would be BM, due to the complications previously described when G-CSF was used to collect autologous peripheral blood stem cells (PBSCs) in SCD patients (Abboud et al. (1998) Lancet 351(9107): 959; Adler et al. (2001) Blood, 97(10): 3313-3314; Fitzhugh et al. (2009) Cytotherapy, 11(4): 464-471). Although general anesthesia imposes a risk for SCD patients as well, current best medical practices can minimize these (Neumayr et al. (1998) Am. J. Hematol. 57(2): 101-108).

The development of integrating vectors for β-globin gene transfer has been challenging due to the complex regulatory elements needed for high-level, erythroid-specific expression (Lisowski and Sadelain (2008) Br. J. Haematol. 141(3): 335-345). γ-Retroviral vectors were unable to transfer these β-globin expression cassettes intact (Gelinas et al. (1989) Adv. Exp. Med. Biol. 271: 135-148; Gelinas et al. (1989) Prog. Clin. Biol. Res. 316B: 235-249). In contrast, lentiviral vectors (LV) can transfer β-globin cassettes intact with relatively high efficiency, although the titers of these vectors are reduced compared with those of vectors bearing simpler cassettes (May et al. (2000) Nature 406(6791): 82-86; Pawliuk et al. (2001) Science, 294(5550): 2368-2371). In the last decade, many groups have developed different β-globin LV for targeting β-hemoglobinopathies, with successful therapeutic results following transplantation of ex vivo-modified HSC in mouse models (May et al. (2000) Nature 406(6791): 82-86; Pawliuk et al. (2001) Science, 294(5550): 2368-2371; Levasseur et al. (2003) Blood, 102(13):4312-4319; Hanawa et al. (2004) Blood, 104(8): 2281-2290; Puthenveetil et al. (2004) Blood, 104(12): 3445-3453; Miccio et al. (2008) Proc. Natl. Acad. Sci. USA, 105(30):10547-10552; Pestina et al. (2008) Mol. Ther. 17(2): 245-252).

Sickle patients with hereditary persistence of fetal hemoglobin (HbF) (HPFH) have improved survival and amelioration of clinical symptoms, with maximal clinical benefits observed when the HbF is elevated above threshold values (e.g., 8%-15% of the total cellular Hb) (Voskaridou et al. (2010) Blood, 115(12): 2354-2363; Platt et al. (1994) N. Engl. J. Med. 330(23): 1639-1644). Therefore, some gene therapy strategies have employed viral vectors carrying the human γ-globin gene (HBG1/2). However, these constructs expressed HbF poorly in adult erythroid cells, since fetal-specific transcription factors are required for high-level expression of the γ-globin gene (Chakalova et al. (2005) Blood 105(5): 2154-2160; Russell (2007) Eur. J. Haematol. 79(6): 516-525). These limitations have been overcome by embedding the exons encoding human γ-globin within the human β-globin gene 5′ promoter and 3′ enhancer elements (Hanawa et al. (2004) Blood, 104(8): 2281-2290; Persons et al. (2002) Blood, 101(6): 2175-2183; Perumbeti et al. (2009) Blood, 114(6): 1174-1185). Breda et al. (2012) PLoS One, 7(3): e32345 used an LV vector encoding the human hemoglobin (HBB) gene to increase the expression of normal HbA in CD34⁺-derived erythroid cells from SCD patients, however, the expression level needed when the HBB gene is used would be higher than would be required for HBG1/2 gene expression to achieve therapeutic benefits in SCD patients.

Another approach is to modify β-globin genes to confer antisickling activity by substituting key amino acids from γ-globin. The modified β-globin cassette should yield the necessary high-level, erythroid-specific expression in adult erythroid cells. Pawliuk et al. (2001) Science, 294(5550): 2368-2371 designed an LV carrying a human β-globin gene with the amino acid modification T87Q. The glutamine at position 87 of γ-globin has been implicated in the anti-sickling activity of HbF (Nagel et al. (1979) Proc. Natl. Acad. Sci., USA, 76(2): 670-672). This anti-sickling construct corrected SCD in 2 murine models of the disease, and a similar LV has been used in a clinical trial for β-thalassemia and SCD in France (Cavazzana-Calvo et al. (2010) Nature, 467(7313): 318-322).

Townes and colleagues have taken a similar approach, developing a recombinant human anti-sickling β-globin gene (HBBAS3) encoding a β-globin protein (HbAS3) that has 3 amino substitutions compared with the original (HbA): T87Q for blocking the lateral contact with the canonical Val 6 of HbS, E22A to disrupt axial contacts (32) and G16D, which confers a competitive advantage over sickle-β-globin chains for interaction with the α-globin polypeptide. Functional analysis of the purified HbAS3 protein demonstrated that this recombinant protein had potent activity to inhibit HbS tetramer polymerization (33). Levasseur et al. (19) showed efficient transduction of BM stem cells from a murine model of SCD with a self-inactivating (SIN) LV carrying the HBBAS3 transgene that resulted in normalized rbc physiology and prevented the pathological manifestations of SCD.

### SUMMARY

The capacity of an improved lentiviral vector carrying the anti-sickling (βAS3) β-globin gene cassette to transduce human BM-derived CD34⁺ cells from SCD donors was characterized, particularly with respect to use in a clinical trial of gene therapy for SCD. The illustrative vector achieved efficient transduction of BM CD34⁺ cells from healthy or SCD donors. The gene expression activity of the vector was assessed at the mRNA and protein levels, the effect of HBBAS3 expression on sickling of deoxygenated rbc was characterized. An in vitro assay detected potential genotoxicity. Transduced BM CD34⁺ cells were also xenografted into immunodeficient mice, and human hematopoietic progenitor cells were re-isolated from the marrow of the mice after 2 to 3 months, subjected to in vitro erythroid differentiation, and found to continue to express the antisickling HBBAS3 gene. These results demonstrate the vector(s) described herein to efficiently transduce SCD BM CD34⁺ progenitor cells and produce sufficient levels of an anti-sickling Hb protein to improve the physiological parameters of the rbc that can be utilized for clinical gene therapy of SCD.

Accordingly, in various aspects, the invention(s) contemplated herein may include, but need not be limited to, any one or more of the following embodiments:

### Embodiment 1

A recombinant lentiviral vector (LV) including an expression cassette comprising a nucleic acid construct including an anti-sickling human beta globin gene encoding an anti-sickling-beta globin polypeptide including the mutations Gly16Asp, Glu22Ala and Thr87Gln, where the LV is a TAT-independent and self-inactivating (SIN) LV.

### Embodiment 2

The vector of embodiment 1, where the anti-sickling human β-globin gene includes about 2.3 kb of recombinant human β-globin gene including exons and introns under the control of the human β-globin gene 5′ promoter and the human β-globin 3′ enhancer.

### Embodiment 3

The vector embodiment 2, where the β-globin gene includes β-globin intron 2 with a 375 bp RsaI deletion from IVS2, and a composite human β-globin locus control region including HS2, HS3, and HS4.

### Embodiment 4

The vector according to any one of embodiments 1-3, further including an insulator in the 3′ LTR.

### Embodiment 5

The vector of embodiment 4, where the insulator includes FB (FII/BEAD-A), a 77 bp insulator element that contains the minimal CTCF binding site enhancer-blocking component of the chicken β-globin 5′ DnaseI-hypersensitive site 4 (5′ HS4) and the analogous region of the human T cell receptor δ/α BEAD-1 insulator (see, e.g., Ramezani et al. (2008) Stem Cell 26: 3257-3266).

### Embodiment 6

The vector of embodiment 4, where the insulator comprises the full length chicken beta-globin HS4 or sub-fragments thereof, and/or the ankyrin gene insulator, and/or other synthetic insulator elements.

### Embodiment 7

The vector according to any one of embodiments 1-6, where the vector includes a ψ region vector genome packaging signal.

### Embodiment 8

The vector according to any one of embodiments 1-7, wherein the 5′ LTR includes a CMV enhancer/promoter.

### Embodiment 9

The vector according to any one of embodiments 1-7, wherein the 5′ LTR includes an CMV, RSV or other strong enhancer/promoter.

### Embodiment 10

The vector according to any one of embodiments 1-9, where the vector includes a Rev Responsive Element (RRE).

### Embodiment 11

The vector according to any one of embodiments 1-10, where the vector includes a central polypurine tract (cPPT).

### Embodiment 12

The vector according to any one of embodiments 1-11, where the vector includes a post-translational regulatory element.

### Embodiment 13

The vector of embodiment 12, wherein the posttranscriptional regulatory element is modified Woodchuck Post-transcriptional Regulatory Element (WPRE).

### Embodiment 14

The vector of embodiment 12, wherein the posttranscriptional regulatory element is hepatitis B virus posttranscriptional regulatory element (HPRE) or other nucleic acid sequences that stabilize the vector-directed RNA transcript.

### Embodiment 15

The vector according to any one of embodiments 1-14, where the vector is incapable of reconstituting a wild-type lentivirus through recombination.

### Embodiment 16

A host cell transduced with a vector according to any one of embodiments 1-15.

### Embodiment 17

The host cell of embodiment 16, wherein the cell is a virus producer cell.

### Embodiment 18

The host cell of embodiment 16, wherein the cell is a stem cell.

### Embodiment 19

The host cell of embodiment 16, where the cell is a stem cell derived from bone marrow (BM).

### Embodiment 20

The host cell of embodiment 16, where the cell is a stem cell derived from cord blood (CB).

### Embodiment 21

The host cell of embodiment 16, where the cell is a stem cell derived from mobilized peripheral blood stem cells (mPBSC).

### Embodiment 22

The host cell of embodiment 16, where the cell is an induced pluripotent stem cell (IPSC).

### Embodiment 23

The host cell of embodiment 16, wherein the cell is a 293T cell.

### Embodiment 24

The host cell of embodiment 16, wherein, wherein the cell is a human hematopoietic progenitor cell.

### Embodiment 25

The host cell of embodiment 24, wherein the human hematopoietic progenitor cell is a CD34⁺ cell.

### Embodiment 26

A method of treating sickle cell disease (SCD) in a subject, where the method involves transducing a stem cell and/or progenitor cell from said subject with a vector according to any one of embodiments 1-15; transplanting said transduced cell or cells derived therefrom into the subject where said cells or derivatives therefrom express said anti-sickling human beta globin gene in an effective amount.

### Embodiment 27

The method of embodiment 26, wherein the cell is a stem cell.

### Embodiment 28

The host cell of embodiment 26, where the cell is a stem cell derived from BM.

### Embodiment 29

The method of embodiment 26, where the cell is a stem cell derived from CB.

### Embodiment 30

The method of embodiment 26, where the cell is a stem cell derived from mobilized peripheral blood stem cells (mPBSC).

### Embodiment 31

The method of embodiment 26, where the cell is an IPSC.

### Embodiment 32

The method of embodiment 26, wherein, wherein the cell is a human hematopoietic progenitor cell.

### Embodiment 33

The method of embodiment 32, wherein the human hematopoietic progenitor cell is a CD34⁺ cell.

### Embodiment 34

A virion comprising and/or produced using a vector according to any one of embodiments 1-15.

### DEFINITIONS

“Recombinant” is used consistently with its usage in the art to refer to a nucleic acid sequence that comprises portions that do not naturally occur together as part of a single sequence or that have been rearranged relative to a naturally occurring sequence. A recombinant nucleic acid is created by a process that involves the hand of man and/or is generated from a nucleic acid that was created by hand of man (e.g., by one or more cycles of replication, amplification, transcription, etc.). A recombinant virus is one that comprises a recombinant nucleic acid. A recombinant cell is one that comprises a recombinant nucleic acid.

As used herein, the term “recombinant lentiviral vector” or “recombinant LV) refers to an artificially created polynucleotide vector assembled from an LV and a plurality of additional segments as a result of human intervention and manipulation.

By “globin nucleic acid molecule” is meant a nucleic acid molecule that encodes a globin polypeptide. In various embodiments the globin nucleic acid molecule may include regulatory sequences upstream and/or downstream of the coding sequence.

By “globin polypeptide” is meant a protein having at least 85%, or at least 90%, or at least 95%, or at least 98% amino acid sequence identity to a human alpha, beta or gamma globin.

The term “therapeutic functional globin gene” refers to a nucleotide sequence the expression of which leads to a globin that does not produce a hemoglobinopathy phenotype, and which is effective to provide therapeutic benefits to an individual with a defective globin gene. The functional globin gene may encode a wild-type globin appropriate for a mammalian individual to be treated, or it may be a mutant form of globin, preferably one which provides for superior properties, for example superior oxygen transport properties or anti-sickling properties. The functional globin gene includes both exons and introns, as well as globin promoters and splice donors/acceptors.

By “an effective amount” is meant the amount of a required agent or composition comprising the agent to ameliorate or eliminate symptoms of a disease relative to an untreated patient. The effective amount of composition(s) used to practice the methods described herein for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

### BRIEF DESCRIPTION OF THE DRAWINGS

FIG. 1 schematically illustrates one embodiment of a LV contemplated herein.

FIG. 2 shows images of rbc without (Panel A) and with (Panel B) gene therapy under conditions that induce sickling.

FIG. 3 illustrates construction of illustrative LVs in accordance with the compositions and methods described herein.

FIGS. 4A-4C. The CCL-βAS3-FB LV provirus carrying the HBBAS3 cassette. FIG. 4A: The CCL-βAS3-FB LV provirus has the HBBAS3 expression cassette with the human β-globin gene exons (arrowheads) with the 3 substitutions to encode the HbAS3 protein, introns, the 3′ and 5′ flanking regions, and the β-globin mini-locus control region (LCR) with hypersensitive sites 2-4. The 3′ LTR contains the SIN deletion and FB insulator, both transferred during reverse transcription (RT) to the 5′ LTR of the proviral DNA. FIG. 4B: To test FB insulator stability, PCR reactions were performed using DNA from cells collected at day 14 of in vitro culture of BM CD34+ cells: mock transduced (lane 1), transduced with the CCL-βAS3 LV (lane 2), and transduced with the CCL-βAS3-FB LV (lane 3). Primers amplified either the 5′ LTR (A to B) or the 3′ LTR (C to D) or the FB insertion sites in both LTRs (A to D) of the provirus. The expected sizes of the PCR products with these primer pairs are indicated for the CCL-βAS3 LV and the CCL-βAS3-FB LV. NTC, no template control. FIG. 4C: CTCF-binding protein ChIP. Chromatin was isolated from K562 cells transduced with the CCL-βAS3-FB LV (FB), the CCL-βAS3-1.2 kb cHS4 LV (cHS4), or the CCL-βAS3 vector lacking the insulator (U3). qPCR amplification was done using primers to the HIV SIN LTR (U3, cHS4, and FB) and to the HIV RRE region of the vector backbone (RRE) as negative control or the cellular c-Myc and H19/ICR sites, known to bind CTCF. *P=0.006. Values shown are mean±SD.

FIGS. 5A-5D. Assessment of transduction and hematopoietic potential of BM CD34⁺ cells in CFU assay and under in vitro erythroid differentiation culture. FIG. 5A: The percentage of plated BM CD34+ cells that grew into hematopoietic colonies by in vitro CFU assay is shown. Values presented are the mean±SD for healthy donor (HD)-mock, n=13; HD-βAS3-FB, n=16; SCD-mock, n=18; and SCD-βAS3-FB, n=24. FIG. 5B: Distribution of hematopoietic colony types formed by BM CD34⁺ cells. The percentages of the different types of hematopoietic colonies identified are represented, following the same patterns as in FIG. 5A. HD-mock, n=5 independent experiments; HD-βAS3-FB, n=7 independent experiments; SCD-mock, n=6 independent experiments; and SCD-βAS3-FB, n=8 independent experiments. Values shown are mean±SD. *P=0.048, by 2-way ANOVA. FIG. 5C: In vitro single CFU grown from transduced SCD CD34+ BM were analyzed for the presence of CCL-βAS3-FB vector provirus and VC/cell by qPCR (n=191 colonies, 5 independent experiments). Graph indicates percentages of the CFU that were negative for vector by qPCR (white, n=134) or that had VC/cell of 1-2 (light gray, n=50), 3-6 (dark gray, n=6), and 7-9 (black, n=1). FIG. 5D: VC/cell for CCL-βAS3-FB-transduced BM CD34⁺ cells grown under in vitro erythroid differentiation culture. Each point represents an independent transduction and culture. BM CD34+ cells were from HD (black circles, n=11) or SCD donors (white squares, n=15). Error bars represent mean values ±SD.

FIGS. 6A-6D. In vitro erythroid differentiation of BM CD34+ cells. FIG. 6A: Fold expansion from BM CD34+ cells grown under in vitro erythroid differentiation conditions over time. The growth curves from a representative experiment are shown. HD-mock, black triangles; HD-βAS3-FB transduced, black circles; SCD-mock, white triangles; SCD-βAS3-FB transduced, white squares. FIG. 6B: Immunophenotypic analysis of CD34⁺ BM SCD-transduced samples during in vitro erythroid culture. Cells were analyzed by flow cytometry for expression of CD34, CD45, CD71, and GpA. Each bar represents the percentage of expression of the indicated surface marker at day 3 (white bars), day 14 (pink bars), and day 21 (red bars). Values shown are mean±SD of 4 independent experiments. Percentage of enucleated rbc was assessed at day 21 (mean±SD of 7 independent experiments) by staining with the DNA dye DRAQ5. FIG. 6C: Flow cytometry analysis of erythroid culture to quantify enucleated rbc. Analysis was made by staining cells with DRAQ5 and antibody to human erythroid marker GpA. Enucleated erythrocytes are present in the left upper quadrant as DRAQ5-negative, GpA-positive cells. FIG. 6D: Photomicrographs of cytocentrifuge preparations from cultures stained by May-Grunwald-Giemsa showing the progression of erythroid differentiation from erythroblast to normoblast at day 8 and 14 to a mostly uniform population of enucleated reticulocytes and erythrocytes at day 21.

FIGS. 7A-7D. HBBAS3 expression after in vitro erythroid differentiation from CD34⁺ BM samples. FIG. 7A: HBBAS3 mRNA expression measured by qRT-PCR from cells transduced to different VC/cell. The percentage of HBBAS3 mRNA achieved from each sample was related to its corresponding VC/cell measured by qPCR. A total of 20 independent transductions are shown. HD, black circles (n=4); SCD, white squares (n=16). FIG. 7B: Representative IEF membrane used to quantify the Hb tetramers present. The left-most lane shows the pI standards of human Hb tetramers from the top down: HbA2, HbS, HbF, and HbA (and the predicted pI for HbAS3). Lanes 1-6 show the IEF of lysates from erythroid cultures initiated with SCD BM CD34⁺ cells, either mock transduced (lane 1) or transduced with the CCL-βAS3-FB LV (lanes 2-6). No HbAS3 protein was detected in the mock-transduced samples (lane 1), while HbAS3 represented of the total Hb the following: 21.78% (lane 2, 1.14 VC), 18.11% (lane 3, 1.08 VC), 19.34% (lane 4, 1.13 VC), 21.34% (lane 5, 0.99 VC), and 20.40% (lane 6, 1.11 VC). Densitometric analyses were used to determine the percentage of HbAS3 of total Hb tetramers, and qPCR was used to measure the VC/cell in the same samples. FIG. 7C: HbAS3 protein produced from cells transduced to different VC/cell (n=10). FIG. 7D: Summary of HBBAS3 expression per VC/cell based on measurement of HBBAS3 mRNA (n=16) and HbAS3 tetramers (protein, n=10). Error bars represent mean values ±SD.

FIGS. 8A-8C. SCD phenotypic correction. FIG. 8A: Phase contrast photomicrographs of deoxygenated erythroid cells. Cells from erythroid differentiation cultures of BM CD34⁺ cells were treated with sodium metabisulfite, and their morphology was assessed using phase contract microscopy. Five examples of sickle rbc are displayed across the top panels, and 5 examples of normal rbc are displayed across the bottom panels. FIG. 8B: Representative field of rbc from mock-transduced SCD CD34⁺ cells (left panel) vs. CCL-βAS3-FB transduced SCD CD34+ cells (right panel) upon deoxygenation with sodium metabisulfite. FIG. 8C: Correlation of the percentage of morphologically “corrected” cells to the VC/cell in each individual culture of CCL-βAS3-FB-transduced SCD BM CD34⁺ cells. The percentage of corrected rbc is defined as the percentage of non-sickled cells in a transduced sample minus the background value of non-sickled cells in the concordant non-transduced sample.

FIG. 9A-9D. In vivo assessment of CCL-βAS3-FB LV transduction of BM CD34+ cells. FIG. 9A: Engraftment of human cells in NSG mice. BM cells isolated from mice from each transplant group (nos. 1-6) were analyzed by flow cytometry to measure the percentage of human CD45⁺ cells among all CD45⁺ cells in the marrow (human and murine) as a measurement of engraftment. Mock transduced, white triangles; CCL-βAS3-FB transduced, black triangles. BM samples from HD were used in transplants 3, 4, and 6 and from SCD donors in transplants 1, 2, and 5. FIG. 9B: Immunophenotypic analysis of human cells isolated from NSG mice transplanted with transduced BM CD34+ cells. Flow cytometry was used to enumerate the percentage of the human CD45+ cells that were positive for the markers of B-lymphoid cells (CD19, white), myeloid progenitors (CD33, light gray), hematopoietic progenitors (CD34, dark gray), and erythroid cells (CD71, black). Mean±SD are shown of 3 independent experiments. Mock, n=4; 13AS3-FB, n=8 mice. FIG. 9C: VC/cell in human cells cultured from NSG mice transplanted with transduced BM CD34⁺ cells. Black circles represent samples from mice transplanted with HD BM, and white squares represent mice transplanted with SCD BM. All the human cells examined from mock-transduced mice were negative for VC analysis by qPCR. FIG. 9D: HBBAS3 mRNA expression measured by qRT-PCR from cells transduced to different VC/cell. Five independent transductions are shown. HD, black circles (n=6); SCD, white squares (n=4).

FIGS. 10A-10C show the results of an assessment of genotoxicity of the CCL-βAS3-FB LV vector. FIG. 10A shows frequency of vector (integration site) IS in and near cancer-associated genes. The bars represent the frequencies of integrations in transcribed regions or within 50 kb of promoters of cancer-associated genes (in vitro, 32.1%; in vivo, 34.3%), as defined in Higgins et al. (44). FIG. 10B shows integration frequency around transcriptional starts sites (TSS). The frequencies of vector IS in the four 5-kb bins in a 20-kb window centered at gene TSS are plotted. The IS are shown for the following: BM CD34⁺ cells analyzed after 2 weeks growth in vitro (lenti in vitro, n=2091; gray bars) and 2-3 months in vivo engraftment in NSG mice (lenti in vivo, n=414; black bars) along with an MLV γ-retroviral vector data set from a clinical gene therapy trial (MLV in vitro, n=828; white bars) (45) and a random data set generated in silico and analyzed by identical methods (random, n=12,837; black line). FIG. 10C: In vitro immortalization (IVIM) assay. The replating frequencies for murine lineage-negative cells transduced with the different vectors are shown, calculated based on Poisson statistics using L-Calc software corrected for the bulk VC/cell measured by qPCR on day 8 pTD. The fractions presented across the lower portion of the figure represent the number of negative assays in which no clones were formed divided by the total number of assays performed for that vector. The horizontal bar represents the mean replating frequency of all positive assays. *P=0.002, by 2-sided Fisher's exact test.

FIGS. 11A-11B. βAS3 LVs plasmid maps and production in the presence or absence of TAT protein. FIG. 11A: Vector plasmid forms of the parental DL-βAS3 (top) in which transcription driven by the HIV-1 enhancer and promoter is dependent upon TAT and the CCL-βAS3-FB (bottom) in which the CMV enhancer/promoter is substituted in the 5′ LTR, eliminating the need for TAT. In both cases, the HIV-1 packaging sequence (Ψ), rev responsive element (RRE), central polypurine tract (cPPT), and the Woodchuck Hepatitis Virus post-transcriptional regulatory element (WPRE) are shown. FIG. 11B: The DL-βAS3, CCL-βAS3, CCL-βAS3-FB and the positive control CCLMND-GFP LV vectors were packaged in the presence (black bars) or absence (white bars) of an HIV-1 TAT expression plasmid. Averages of three experiments and SD are shown.

FIG. 12. Southern Blot analysis was performed to confirm full length integrity of the provirus. Genomic DNA of 293T cells, mock-transduced or transduced with the CCL-βAS3-FB LV (with an average VC/cell of 10 analyzed by qPCR) was digested by AflII, which cuts in each LTR of the provirus and should release a nearly full-length genome fragment (8.6 Kb). The DNA ladder is shown in the lane 1, followed by the mock-transduced cells in lane 2 and the CCL-βAS3-FB-transduced cells in the lane 3, where a unique band representing the intact provirus of the right size is present.

FIG. 13. HBBAS3 mRNA expression at day 14 of erythroid or myeloid cultures was analyzed relative to the endogenous control gene ACTB. In three separate experiments, no mRNA expression by the HBBAS3 transgene was detected in myeloid conditions (0.04±0.01) relative expression compared to ACTB. In contrast, the same cells grown under erythroid conditions, showed high expression of HBBAS3 mRNA (235.35±77.77). The mRNA expression in each condition was normalized to the VC/cell obtained from the erythroid and myeloid samples, respectively. Values shown are average ±SD.

FIG. 14. Expression of the HBBAS3 cassette from erythroid cells produced by BM-CD34⁺ cells from SCD donors, transduced with the CCL-βAS3 or the CCL-βAS3-FB LV, was analyzed by RTqPCR to determine the percentage of HBBAS3 mRNA per VC/cell (solid rhombus); or by IEF to determine the percentage of HbAS3 protein per VC/cell (empty rhombus). No differences were found in the percentage of HBBAS3 mRNA of the total beta-globin-like mRNA (p=0.12, twotailed t-test); or in the percentage of HbAS3 of the total Hb (p=0.89, two-tailed t-test) in erythroid cells transduced with the CCL-βAS3 or the CCL-βAS3-FB LV. Therefore, these results indicate that the FB insulator did not provide barrier activity to improve position-independent expression; since the addition of the FB insulator did not alter the expression of the HBBAS3 cassette when compared to the non-insulated LV. Error bars represent mean values.

FIG. 15 shows VC/cell determined by qPCR in CCL-βAS3-FB-transduced BM CD34⁺ cells grown in erythroid conditions, methylcellulose medium (CFU), myeloid conditions and expanded from engrafted NSG BM. VC/cell measurements from cells grown in erythroid culture assay were significantly higher than those measured in cells grown in myeloid culture (*p=0.0003) or from NSG BM (**p<0.0001). Values shown are average ±SD.

FIG. 16 schematically illustrates typical steps in cell based gene therapy of sickle disease.

### DETAILED DESCRIPTION

Sickle cell disease (SCD) is a multisystem disease, associated with severe episodes of acute illness and progressive organ damage, and is one of the most common monogenic disorders worldwide. Because SCD results from abnormalities in rbc, which in turn are produced from adult HSC, HSCT from a healthy (allogeneic) donor can benefit patients with SCD, by providing a source for life-long production of normal red blood cells. However, allogeneic HSCT is limited by the availability of well-matched donors and by immunological complications of graft rejection and graft-versus-host disease.

We believe that autologous stem cell gene therapy for SCD has the potential to treat this illness without the need for immune suppression of current allogeneic HSCT approaches. In particular, we believe that autologous stem cell gene therapy that introduces anti-sickling human beta globin into hematopoietic cells (or progenitors thereof) can provide effective therapy for SCD (including, for example, normalized rbc physiology and prevention of the manifestations of SCD).

Accordingly, in various embodiments, an improved LV is provided for the introduction of anti-sickling beta globin into stem and progenitor cells (e.g., hematopoietic stem and progenitor cells) that can then be transplanted into a subject in need thereof (e.g., a subject that has the sickle cell mutation). In certain embodiments the anti-sickling version of a human beta globin gene used in the vector comprises three mutations Gly16Asp, Glu22Ala and Thr87Gln (see, e.g., Levasseur (2004) J. Biol. Chem. 279(26): 27518-27524). Without being bound to a particular theory, it is believed the Glu22Ala mutation increases affinity to α-chain, the Thr87Gln mutation blocks lateral contact with Val6 of βS protein, and the Gly16Asp mutation decreases axial contact between globin chains.

In various embodiments, the LVs described herein have additional safety features not included in previous β-globin encoding lentiviral constructs. In certain embodiments, these features include the presence of an insulator (e.g., an FB insulator in the 3′LTR). Additionally or alternatively, in certain embodiments, the HIV LTR has been substituted with an alternative promoter (e.g., a CMV) to yield a higher titer vector without the inclusion of the HIV TAT protein during packaging. Other strong promoters (e.g., RSV, and the like can also be used).

Additionally, as explained below, the efficacy of the vectors described herein using HSC from the BM of patients with SCD have also been demonstrated for the first time.

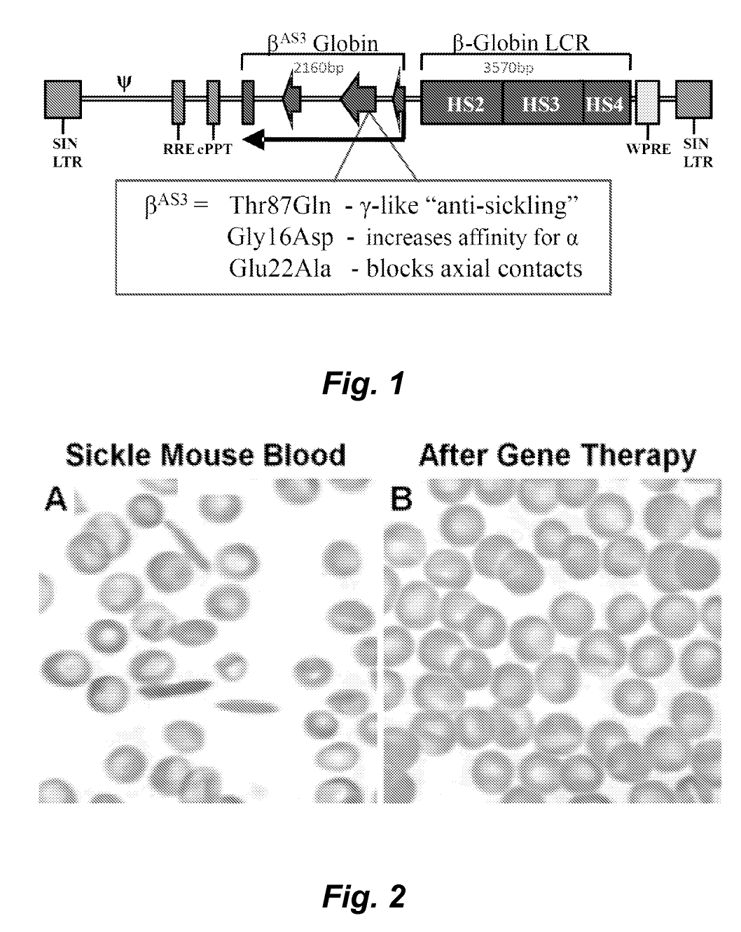

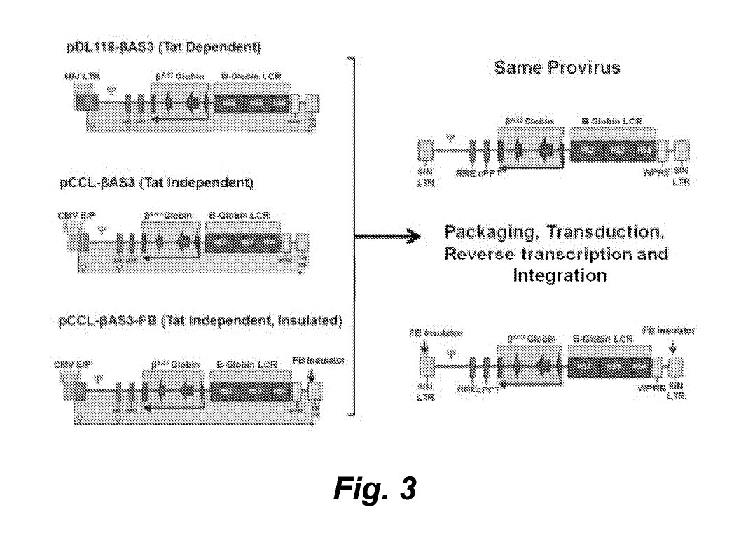

As proof of principle, a LV was fabricated comprising the βAS3 globin expression cassette inserted into the pCCL LV vector backbone to confer tat-independence for packaging (see, e.g., FIGS. 1, 3, 4A, and 4B illustrating various vectors and assembly strategy). In certain embodiments the FB (FII/BEAD-A) composite enhancer-blocking insulator (Ramezani et al. (2008) Stem Cell 26: 3257-3266) was inserted into the 3′ LTR providing the βAS3-FB LV.

Assessments were performed by transducing human BM CD34⁺ cells from healthy or SCD donors with βAS3 LV vectors. Efficient (0.5-2 vector copies/cell) and stable gene transmission were determined by qPCR and Southern Blot.

CFU assays showed that these cells were fully capable of maintaining their hematopoietic potential and that 31±4% were transduced based on qPCR analysis. To determine the effectiveness of the erythroid-specific βAS3 cassette in the context of human Hematopoietic Stem and Progenitor Cells (huHSPC), we optimized an in vitro model of erythroid differentiation. We obtained an expansion up to 700 fold with >80% fully mature enucleated rbc derived from CD34⁺ cells from SCD and HD.

From the rbc derived from the SCD BM CD34+ transduced cells, βAS3 globin gene expression was analyzed by isoelectric focusing (IEF), obtaining an average of 18% HbAS3 over the total globin produced, per Vector Copy Number (VCN). βAS3 mRNA expression in transduced cells was analyzed by a qRT-PCR assay able to discriminate β^{AS3} vs. β and β^{S} transcripts respectively, confirming the quantitative expression results obtained by IEF. We also demonstrated morphological correction of in vitro differentiated rbc from SCD BM CD34+ cell transduced with the CCL-βAS3-FB LV. Upon induction of deoxygenation, 42% fewer cells showed sickle shape in the samples modified with the β^{AS3} gene vs. the non-transduced ones (see, e.g., FIG. 2).

Finally, we performed in vivo studies. After transplanting BM CD34⁺ cells from SCD and HD transduced with the CCL-βAS3-FB LV in NSG mice we were able to detect an average of 19% βAS3 mRNA of the total β-like transcripts per VC. Preliminary results from our approach to assess vector safety indicate the lack of insertional transformation in murine hematopoietic stem and progenitor cells transduced with CCL-βAS3-FB LV. These results demonstrate that βAS3-FB LV is capable of efficient transfer and sufficient expression of an anti-sickling β-globin gene to CD34⁺ progenitor cells leading to improved physiologic parameters of the mature rbc.

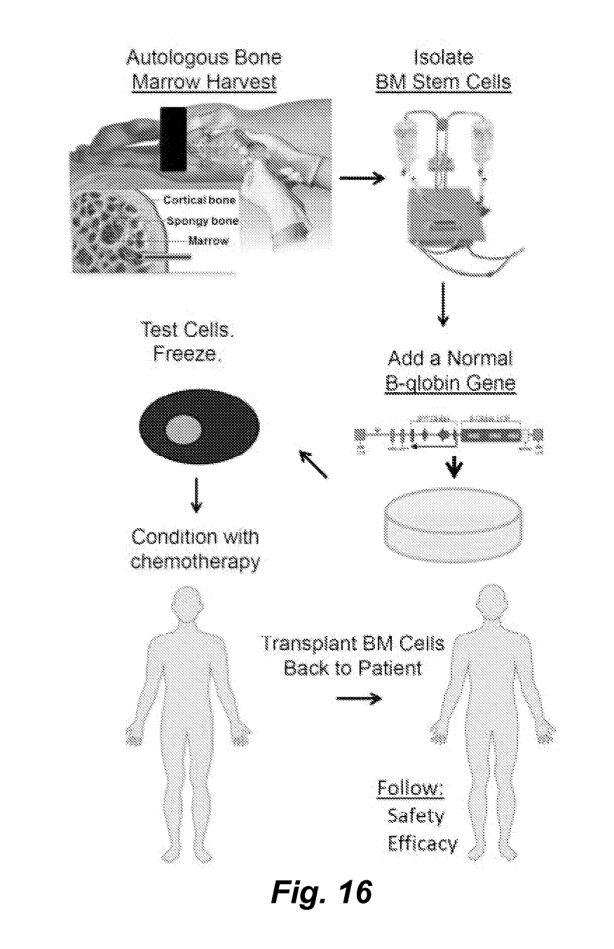

In view of these results, it is believed that LVs described herein, e.g., recombinant TAT-independent, SIN LVs that express an anti-sickling human beta globin can be used to effectively treat subjects with SCD (e.g., subjects that have the sickle cell mutation). It is believed these vectors can be used for the modification of stem cells (e.g., hematopoietic stem and progenitor cells) that can be introduced into a subject in need thereof for the treatment of SCD (e.g., as illustrated in FIG. 16). Moreover, it appears that the resulting cells will produce enough of the transgenic β-globin protein to demonstrate significant improvement in subject health. It is also believed the vectors can be directly administered to a subject to achieve in vivo transduction of the target (e.g., hematopoietic stem or progenitor cells) and thereby also effect a treatment of subjects in need thereof.

As noted above, in various embodiments, the LVs described herein comprise safety features not included in the previous vectors of this type. In particular, the HIV LTR has been substituted with a CMV promoter to yield higher titer vector without the inclusion of the HIV TAT protein during packaging. In certain embodiments an insulator (e.g., the FB insulator) is introduced into the 3′LTR for safety. The LVs are also constructed to provide efficient transduction and high titer.

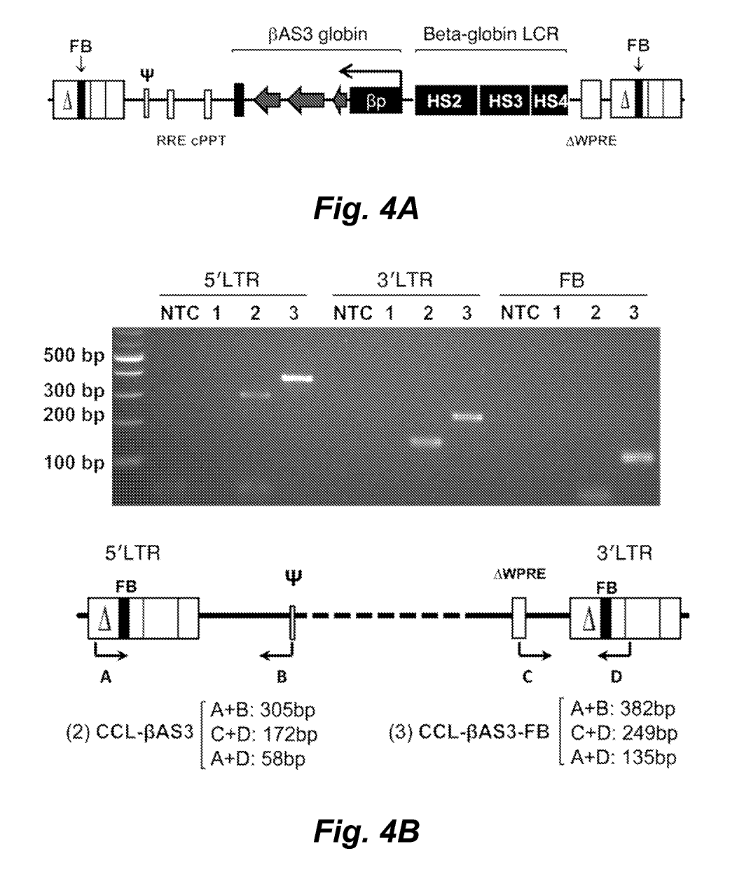

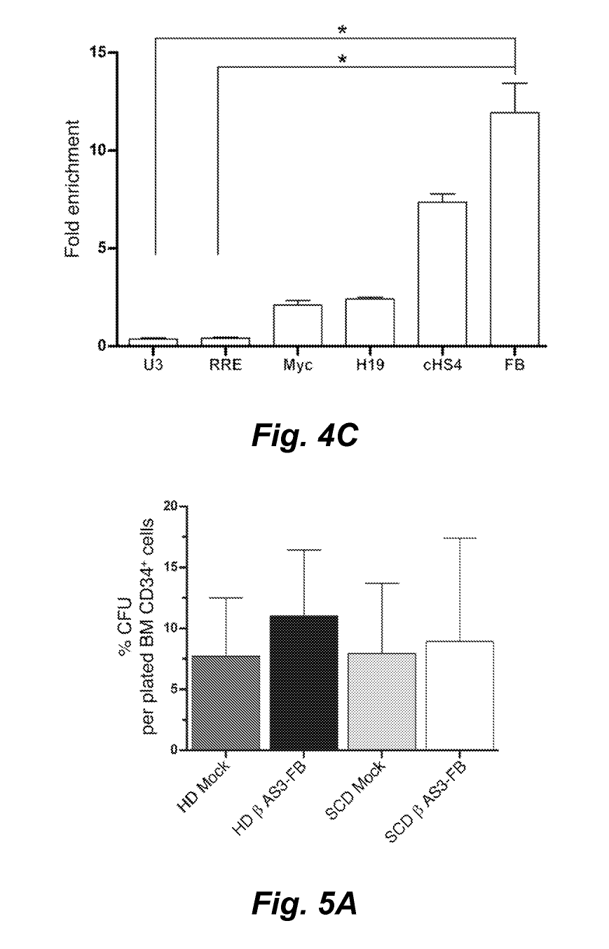

In certain embodiments (see, e.g., FIGS. 1, 4A, and 4B), the components of the vector comprise at least elements 1 and 2 below, or at least elements 1, 2, and 4 below, or at least elements 1, 2, 4, and 5 below, or at least elements 1, 2, 4, 5, and 6 below, or at least elements 1, 2, 4, 5, and 6 below, or at least elements 1, 2, 4, 5, 6, and 7 below, or at least elements 1, 2, 3, 4, 5, 6, and 7 below:
1) An expression cassette encoding an anti-sickling human β-globin (e.g., βAS3);
2) A self-inactivating (SIN) LTR configuration;
3) An (optional) insulator element (e.g., FB);
4) A packaging signal (e.g., Ψ);
5) A Rev Responsive Element (RRE) to enhance nuclear export of unspliced vector RNA;
6) A central polypurine tract (cPPT) to enhance nuclear import of vector genomes; and
7) A post-transcriptional regulatory element (PRE) to enhance vector genome stability and to improve vector titers (e.g., WPRE).

It will be appreciated that the foregoing elements are illustrative and need not be limiting. In view of the teachings provided herein, suitable substitutions for these elements will be recognized by one of skill in the art and are contemplated within the scope of the teachings provided herein.

### Anti-Sickling Beta Globin Gene and Expression Cassette.

As indicated above, in various embodiments the LV described herein comprise an expression cassette encoding an anti-sickling human β-globin gene. On illustrative, but non-limiting cassette is βAS3 which comprises an ˜2.3 kb recombinant human β-globin gene (exons and introns) with three amino acid substitutions (Thr87Gln; Gly16Asp; and Glu22Ala) under the control of transcriptional control elements (e.g., the human β-globin gene 5′ promoter (e.g., ˜266 bp), the human β-globin 3′ enhancer (e.g., ˜260 bp), β-globin intron 2 with a ˜375 bp RsaI deletion from IVS2, and a ˜3.4 kb composite human β-globin locus control region (e.g., HS2˜1203 bp; HS3˜1213 bp; HS4˜954 bp). One embodiment of a βAS3 cassette is described by Levasseur (2003) Blood 102: 4312-4319.

The βAS3 cassette, however, is illustrative and need not be limiting. Using the known cassette described herein (see, e.g., Example 1), numerous variations will be available to one of skill in the art. Such variations include, for example, further and/or alternative mutations to the β-globin to further enhance non-sickling properties, alterations in the transcriptional control elements (e.g., promoter and/or enhancer), variations on the intron size/structure, and the like.

### TAT-Independent and Self Inactivating Lentiviral Vectors.

To further improve safety, in various embodiments, the LVs described herein comprise a TAT-independent, self-inactivating (SIN) configuration. Thus, in various embodiments it is desirable to employ in the LVs described herein an LTR region that has reduced promoter activity relative to wild-type LTR. Such constructs can be provided that are effectively “self-inactivating” (SIN) which provides a biosafety feature. SIN vectors are ones in which the production of full-length vector RNA in transduced cells is greatly reduced or abolished altogether. This feature minimizes the risk that replication-competent recombinants (RCRs) will emerge. Furthermore, it reduces the risk that that cellular coding sequences located adjacent to the vector integration site will be aberrantly expressed.

Furthermore, a SIN design reduces the possibility of interference between the LTR and the promoter that is driving the expression of the transgene. SIN LVs can often permit full activity of the internal promoter.

The SIN design increases the biosafety of the LVs. The majority of the HIV LTR is comprised of the U3 sequences. The U3 region contains the enhancer and promoter elements that modulate basal and induced expression of the HIV genome in infected cells and in response to cell activation. Several of these promoter elements are essential for viral replication. Some of the enhancer elements are highly conserved among viral isolates and have been implicated as critical virulence factors in viral pathogenesis. The enhancer elements may act to influence replication rates in the different cellular target of the virus

As viral transcription starts at the 3′ end of the U3 region of the 5′ LTR, those sequences are not part of the viral mRNA and a copy thereof from the 3′ LTR acts as template for the generation of both LTR's in the integrated provirus. If the 3′ copy of the U3 region is altered in a retroviral vector construct, the vector RNA is still produced from the intact 5′ LTR in producer cells, but cannot be regenerated in target cells. Transduction of such a vector results in the inactivation of both LTR's in the progeny virus. Thus, the retrovirus is self-inactivating (SIN) and those vectors are known as SIN transfer vectors.

In certain embodiments self-inactivation is achieved through the introduction of a deletion in the U3 region of the 3′ LTR of the vector DNA, i.e., the DNA used to produce the vector RNA. During RT, this deletion is transferred to the 5′ LTR of the proviral DNA. Typically, it is desirable to eliminate enough of the U3 sequence to greatly diminish or abolish altogether the transcriptional activity of the LTR, thereby greatly diminishing or abolishing the production of full-length vector RNA in transduced cells. However, it is generally desirable to retain those elements of the LTR that are involved in polyadenylation of the viral RNA, a function typically spread out over U3, R and U5. Accordingly, in certain embodiments, it is desirable to eliminate as many of the transcriptionally important motifs from the LTR as possible while sparing the polyadenylation determinants.

The SIN design is described in detail in Zufferey et al. (1998) J Virol. 72(12): 9873-9880, and in U.S. Pat. No. 5,994,136. As described therein, there are, however, limits to the extent of the deletion at the 3′ LTR. First, the 5′ end of the U3 region serves another essential function in vector transfer, being required for integration (terminal dinucleotide+att sequence). Thus, the terminal dinucleotide and the att sequence may represent the 5′ boundary of the U3 sequences which can be deleted. In addition, some loosely defined regions may influence the activity of the downstream polyadenylation site in the R region. Excessive deletion of U3 sequence from the 3′LTR may decrease polyadenylation of vector transcripts with adverse consequences both on the titer of the vector in producer cells and the transgene expression in target cells.

Additional SIN designs are described in U.S. Patent Publication No: 2003/0039636. As described therein, in certain embodiments, the lentiviral sequences removed from the LTRs are replaced with comparable sequences from a non-lentiviral retrovirus, thereby forming hybrid LTRs. In particular, the lentiviral R region within the LTR can be replaced in whole or in part by the R region from a non-lentiviral retrovirus. In certain embodiments, the lentiviral TAR sequence, a sequence which interacts with TAT protein to enhance viral replication, is removed, preferably in whole, from the R region. The TAR sequence is then replaced with a comparable portion of the R region from a non-lentiviral retrovirus, thereby forming a hybrid R region. The LTRs can be further modified to remove and/or replace with non-lentiviral sequences all or a portion of the lentiviral U3 and U5 regions.

Accordingly, in certain embodiments, the SIN configuration provides a retroviral LTR comprising a hybrid lentiviral R region that lacks all or a portion of its TAR sequence, thereby eliminating any possible activation by TAT, wherein the TAR sequence or portion thereof is replaced by a comparable portion of the R region from a non-lentiviral retrovirus, thereby forming a hybrid R region. In a particular embodiment, the retroviral LTR comprises a hybrid R region, wherein the hybrid R region comprises a portion of the HIV R region (e.g., a portion comprising or consisting of the nucleotide sequence shown in SEQ ID NO: 10 in US 2003/0039636) lacking the TAR sequence, and a portion of the MoMSV R region (e.g., a portion comprising or consisting of the nucleotide sequence shown in SEQ ID NO: 9 in 2003/0039636) comparable to the TAR sequence lacking from the HIV R region. In another particular embodiment, the entire hybrid R region comprises or consists of the nucleotide sequence shown in SEQ ID NO: 11 in 2003/0039636.

Suitable lentiviruses from which the R region can be derived include, for example, HIV (HIV-1 and HIV-2), EIV, SIV and FIV. Suitable retroviruses from which non-lentiviral sequences can be derived include, for example, MoMSV, MoMLV, Friend, MSCV, RSV and Spumaviruses. In one illustrative embodiment, the lentivirus is HIV and the non-lentiviral retrovirus is MoMSV.

In another embodiment described in US 2003/0039636, the LTR comprising a hybrid R region is a left (5′) LTR and further comprises a promoter sequence upstream from the hybrid R region. Preferred promoters are non-lentiviral in origin and include, for example, the U3 region from a non-lentiviral retrovirus (e.g., the MoMSV U3 region). In one particular embodiment, the U3 region comprises the nucleotide sequence shown in SEQ ID NO: 12 in US 2003/0039636. In another embodiment, the left (5′) LTR further comprises a lentiviral U5 region downstream from the hybrid R region. In one embodiment, the U5 region is the HIV U5 region including the HIV att site necessary for genomic integration. In another embodiment, the U5 region comprises the nucleotide sequence shown in SEQ ID NO: 13 in US 2003/0039636. In yet another embodiment, the entire left (5′) hybrid LTR comprises the nucleotide sequence shown in SEQ ID NO: 1 in US 2003/0039636.

In another illustrative embodiment, the LTR comprising a hybrid R region is a right (3′) LTR and further comprises a modified (e.g., truncated) lentiviral U3 region upstream from the hybrid R region. The modified lentiviral U3 region can include the att sequence, but lack any sequences having promoter activity, thereby causing the vector to be SIN in that viral transcription cannot go beyond the first round of replication following chromosomal integration. In a particular embodiment, the modified lentiviral U3 region upstream from the hybrid R region consists of the 3′ end of a lentiviral (e.g., HIV) U3 region up to and including the lentiviral U3 att site. In one embodiment, the U3 region comprises the nucleotide sequence shown in SEQ ID NO: 15 in US 2003/0039636. In another embodiment, the right (3′) LTR further comprises a polyadenylation sequence downstream from the hybrid R region. In another embodiment, the polyadenylation sequence comprises the nucleotide sequence shown in SEQ ID NO: 16 in US 2003/0039636. In yet another embodiment, the entire right (5′) LTR comprises the nucleotide sequence shown in SEQ ID NO: 2 or 17 of US 2003/0039636.

Thus, in the case of HIV based LV, it has been discovered that such vectors tolerate significant U3 deletions, including the removal of the LTR TATA box (e.g., deletions from −418 to −18), without significant reductions in vector titers. These deletions render the LTR region substantially transcriptionally inactive in that the transcriptional ability of the LTR in reduced to about 90% or lower.

It has also been demonstrated that the trans-acting function of Tat becomes dispensable if part of the upstream LTR in the transfer vector construct is replaced by constitutively active promoter sequences (see, e.g., Dull et al. (1998) J Virol. 72(11): 8463-8471. Furthermore, we show that the expression of rev in trans allows the production of high-titer HIV-derived vector stocks from a packaging construct which contains only gag and pol. This design makes the expression of the packaging functions conditional on complementation available only in producer cells. The resulting gene delivery system, conserves only three of the nine genes of HIV-1 and relies on four separate transcriptional units for the production of transducing particles.

In one embodiments illustrated in Example 1, the cassette expressing an anti-sickling β-globin (e.g., βAS3) is placed in the pCCL LV backbone, which is a SIN vector with the CMV enhancer/promoter substituted in the 5′ LTR.

It will be recognized that the CMV promoter typically provides a high level of non-tissue specific expression. Other promoters with similar constitutive activity include, but are not limited to the RSV promoter, and the SV40 promoter. Mammalian promoters such as the beta-actin promoter, ubiquitin C promoter, elongation factor 1αpromoter, tubulin promoter, etc., may also be used.

The foregoing SIN configurations are illustrative and non-limiting. Numerous SIN configurations are known to those of skill in the art. As indicated above, in certain embodiments, the LTR transcription is reduced by about 95% to about 99%. In certain embodiments LTR may be rendered at least about 90%, at least about 91%, at least about 92%, at least about 93%, at least about 94%, at least about 95% at least about 96%, at least about 97%, at least about 98%, or at least about 99% transcriptionally inactive.

### Insulator Element

In certain embodiments, to further enhance biosafety, insulators are inserted into the LV described herein. Insulators are DNA sequence elements present throughout the genome. They bind proteins that modify chromatin and alter regional gene expression. The placement of insulators in the vectors described herein offer various potential benefits including, inter alia: 1) Shielding of the vector from positional effect variegation of expression by flanking chromosomes (i.e., barrier activity); and 2) Shielding flanking chromosomes from insertional trans-activation of gene expression by the vector (enhancer blocking) Thus, insulators can help to preserve the independent function of genes or transcription units embedded in a genome or genetic context in which their expression may otherwise be influenced by regulatory signals within the genome or genetic context (see, e.g., Burgess-Beusse et al. (2002) Proc. Natl. Acad. Sci. USA, 99: 16433; and Zhan et al. (2001) Hum. Genet., 109: 471). In the present context insulators may contribute to protecting lentivirus-expressed sequences from integration site effects, which may be mediated by cis-acting elements present in genomic DNA and lead to deregulated expression of transferred sequences. In various embodiments LVs are provided in which an insulator sequence is inserted into one or both LTRs or elsewhere in the region of the vector that integrates into the cellular genome.

The first and best characterized vertebrate chromatin insulator is located within the chicken β-globin locus control region. This element, which contains a DNase-I hypersensitive site-4 (cHS4), appears to constitute the 5′ boundary of the chicken β-globin locus (Prioleau et al. (1999) EMBO J. 18: 4035-4048). A 1.2-kb fragment containing the cHS4 element displays classic insulator activities, including the ability to block the interaction of globin gene promoters and enhancers in cell lines (Chung et al. (1993) Cell, 74: 505-514), and the ability to protect expression cassettes in Drosophila (Id.), transformed cell lines (Pikaart et al. (1998) Genes Dev. 12: 2852-2862), and transgenic mammals (Wang et al. (1997) Nat. Biotechnol., 15: 239-243; Taboit-Dameron et al. (1999) Transgenic Res., 8: 223-235) from position effects. Much of this activity is contained in a 250-bp fragment. Within this stretch is a 49-bp cHS4 core (Chung et al. (1997) Proc. Natl. Acad. Sci., USA, 94: 575-580) that interacts with the zinc finger DNA binding protein CTCF implicated in enhancer-blocking assays (Bell et al. (1999) Cell, 98: 387-396).

One illustrative and suitable insulator is FB (FII/BEAD-A), a 77 bp insulator element, that contains the minimal CTCF binding site enhancer-blocking components of the chicken β-globin 5′ HS4 insulators and a homologous region from the human T-cell receptor alpha/delta blocking element alpha/delta I (BEAD-I) insulator described by Ramezani et al. (2008) Stem Cell 26: 3257-3266. The FB “synthetic” insulator has full enhancer blocking activity. This insulator is illustrative and non-limiting. Other suitable insulators may be used including, for example, the full length chicken beta-globin HS4 or insulator sub-fragments thereof, the ankyrin gene insulator, and other synthetic insulator elements.

### Packaging Signal.

In various embodiments the vectors described herein further comprise a packaging signal. A “packaging signal,” “packaging sequence,” or “psi sequence” is any nucleic acid sequence sufficient to direct packaging of a nucleic acid whose sequence comprises the packaging signal into a retroviral particle. The term includes naturally occurring packaging sequences and also engineered variants thereof. Packaging signals of a number of different retroviruses, including lentiviruses, are known in the art.

### Rev Responsive Element (RRE).

In certain embodiments the LVs described herein comprise a Rev response element (RRE) to enhance nuclear export of unspliced RNA. RREs are well known to those of skill in the art. Illustrative RREs include, but are not limited to RREs such as that located at positions 7622-8459 in the HIV NL4-3 genome (Genbank accession number AF003887) as well as RREs from other strains of HIV or other retroviruses. Such sequences are readily available from Genbank or from the database with URL hiv-web.lanl.gov/content/index.

Central PolyPurine Tract (cPPT).

In various embodiments the vectors described herein further include a central polypurine tract. Insertion of a fragment containing the central polypurine tract (cPPT) in lentiviral (e.g., HIV-1) vector constructs is known to enhance transduction efficiency drastically, reportedly by facilitating the nuclear import of viral cDNA through a central DNA flap.

### Expression-Stimulating Posttranscriptional Regulatory Element (PRE)

In certain embodiments the LVs described herein may comprise any of a variety of posttranscriptional regulatory elements (PREs) whose presence within a transcript increases expression of the heterologous nucleic acid (e.g., βAS3) at the protein level. PREs may be particularly useful in certain embodiments, especially those that involve lentiviral constructs with modest promoters.

One type of PRE is an intron positioned within the expression cassette, which can stimulate gene expression. However, introns can be spliced out during the life cycle events of a lentivirus. Hence, if introns are used as PRE's they are typically placed in an opposite orientation to the vector genomic transcript.

Posttranscriptional regulatory elements that do not rely on splicing events offer the advantage of not being removed during the viral life cycle. Some examples are the posttranscriptional processing element of herpes simplex virus, the posttranscriptional regulatory element of the hepatitis B virus (HPRE) and the woodchuck hepatitis virus (WPRE). Of these the WPRE is typically preferred as it contains an additional cis-acting element not found in the HPRE. This regulatory element is typically positioned within the vector so as to be included in the RNA transcript of the transgene, but outside of stop codon of the transgene translational unit.

The WPRE is characterized and described in U.S. Pat. No. 6,136,597. As described therein, the WPRE is an RNA export element that mediates efficient transport of RNA from the nucleus to the cytoplasm. It enhances the expression of transgenes by insertion of a cis-acting nucleic acid sequence, such that the element and the transgene are contained within a single transcript. Presence of the WPRE in the sense orientation was shown to increase transgene expression by up to 7 to 10 fold. Retroviral vectors transfer sequences in the form of cDNAs instead of complete intron-containing genes as introns are generally spliced out during the sequence of events leading to the formation of the retroviral particle. Introns mediate the interaction of primary transcripts with the splicing machinery. Because the processing of RNAs by the splicing machinery facilitates their cytoplasmic export, due to a coupling between the splicing and transport machineries, cDNAs are often inefficiently expressed. Thus, the inclusion of the WPRE in a vector results in enhanced expression of transgenes.

### Transduced Host Cells and Methods of Cell Transduction.

The recombinant LV and resulting virus described herein are capable of transferring a nucleic acid (e.g., a nucleic acid encoding an anti-sickling β-globin) sequence into a mammalian cell. For delivery to cells, vectors of the present invention are preferably used in conjunction with a suitable packaging cell line or co-transfected into cells in vitro along with other vector plasmids containing the necessary retroviral genes (e.g., gag and pol) to form replication incompetent virions capable of packaging the vectors of the present invention and infecting cells.

The recombinant LVs and resulting virus described herein are capable of transferring a nucleic acid (e.g., a nucleic acid encoding an anti-sickling β-globin) sequence into a mammalian cell. For delivery to cells, vectors of the present invention are preferably used in conjunction with a suitable packaging cell line or co-transfected into cells in vitro along with other vector plasmids containing the necessary retroviral genes (e.g., gag and pol) to form replication incompetent virions capable of packaging the vectors of the present invention and infecting cells.

Typically, the vectors are introduced via transfection into the packaging cell line. The packaging cell line produces viral particles that contain the vector genome. Methods for transfection are well known by those of skill in the art. After cotransfection of the packaging vectors and the transfer vector to the packaging cell line, the recombinant virus is recovered from the culture media and tittered by standard methods used by those of skill in the art. Thus, the packaging constructs can be introduced into human cell lines by calcium phosphate transfection, lipofection or electroporation, generally together with a dominant selectable marker, such as neomycin, DHFR, Glutamine synthetase, followed by selection in the presence of the appropriate drug and isolation of clones. In certain embodiments the selectable marker gene can be linked physically to the packaging genes in the construct.

Stable cell lines wherein the packaging functions are configured to be expressed by a suitable packaging cell are known (see, e.g., U.S. Pat. No. 5,686,279, which describes packaging cells). In general, for the production of virus particles, one may employ any cell that is compatible with the expression of lentiviral Gag and Pol genes, or any cell that can be engineered to support such expression. For example, producer cells such as 293T cells and HT1080 cells may be used.

The packaging cells with a lentiviral vector incorporated in them form producer cells. Producer cells are thus cells or cell-lines that can produce or release packaged infectious viral particles carrying the therapeutic gene of interest (e.g., modified β-globin). These cells can further be anchorage dependent which means that these cells will grow, survive, or maintain function optimally when attached to a surface such as glass or plastic. Some examples of anchorage dependent cell lines used as lentiviral vector packaging cell lines when the vector is replication competent are HeLa or 293 cells and PERC.6 cells.

Accordingly, in certain embodiments, methods are provided of delivering a gene to a cell which is then integrated into the genome of the cell, comprising contacting the cell with a virion containing a lentiviral vector described herein. The cell (e.g., in the form of tissue or an organ) can be contacted (e.g., infected) with the virion ex vivo and then delivered to a subject (e.g., a mammal, animal or human) in which the gene (e.g., anti-sickling β-globin) will be expressed. In various embodiments the cell can be autologous to the subject (i.e., from the subject) or it can be non-autologous (i.e., allogeneic or xenogenic) to the subject. Moreover, because the vectors described herein are capable of being delivered to both dividing and non-dividing cells, the cells can be from a wide variety including, for example, bone marrow cells, mesenchymal stem cells (e.g., obtained from adipose tissue), and other primary cells derived from human and animal sources. Alternatively, the virion can be directly administered in vivo to a subject or a localized area of a subject (e.g., bone marrow).

Of course, as noted above, the lentivectors described herein will be particularly useful in the transduction of human hematopoietic progenitor cells or a hematopoietic stem cells, obtained either from the bone marrow, the peripheral blood or the umbilical cord blood, as well as in the transduction of a CD4⁺ T cell, a peripheral blood B or T lymphocyte cell, and the like. In certain embodiments particularly preferred targets are CD34⁺ cells.

### Gene Therapy.

In still other embodiments, the present invention is directed to a method for transducing a human hematopoietic stem cell comprising contacting a population of human cells that include hematopoietic stem cells with one of the foregoing lentivectors under conditions to effect the transduction of a human hematopoietic progenitor cell in said population by the vector. The stem cells may be transduced in vivo or in vitro, depending on the ultimate application. Even in the context of human gene therapy, such as gene therapy of human stem cells, one may transduce the stem cell in vivo or, alternatively, transduce in vitro followed by infusion of the transduced stem cell into a human subject. In one aspect of this embodiment, the human stem cell can be removed from a human, e.g., a human patient, using methods well known to those of skill in the art and transduced as noted above. The transduced stem cells are then reintroduced into the same or a different human.

Stem Cell/Progenitor Cell Gene Therapy.

In various embodiments the lentivectors described herein are particularly useful for the transduction of human hematopoietic progenitor cells or haematopoietic stem cells (HSCs), obtained either from the bone marrow, the peripheral blood or the umbilical cord blood, as well as in the transduction of a CD4⁺ T cell, a peripheral blood B or T lymphocyte cell, and the like. In certain embodiments particularly preferred targets are CD34⁺ cells.

When cells, for instance CD34⁺ cells, dendritic cells, peripheral blood cells or tumor cells are transduced ex vivo, the vector particles are incubated with the cells using a dose generally in the order of between 1 to 50 multiplicities of infection (MOI) which also corresponds to 1×10⁵ to 50×10⁵ transducing units of the viral vector per 10⁵ cells. This of course includes amount of vector corresponding to 1, 2, 3, 4, 5, 6, 7, 8, 9, 10, 15, 20, 25, 30, 35, 40, 45, and 50 MOI. Typically, the amount of vector may be expressed in terms of HeLa transducing units (TU).

It is noted that as shown in Example 1, a dose-related increase in gene transfer achieved (the average VC/cell measured by qPCR) was found only for vector concentrations below 2×10⁷ TU/ml. Higher vector concentrations did not increase the transduction efficacy and, in fact, often had a negative effect on the extent of transduction (data not shown). Based on these findings, the CCL-βAS3-FB vector was used at a standard concentration of 2×10⁷ TU/ml (MOI=40).

In certain embodiments cell-based therapies involve providing stem cells and/or hematopoietic precursors, transduce the cells with the lentivirus encoding an anti-sickling human β-globin, and then introduce the transformed cells into a subject in need thereof (e.g., a subject with the sickle cell mutation).

In certain embodiments the methods involve isolating population of cells, e.g., stem cells from a subject, optionally expand the cells in tissue culture, and administer the lentiviral vector whose presence within a cell results in production of an anti-sickling β-globin in the cells in vitro. The cells are then returned to the subject, where, for example, they may provide a population of red blood cells that produce the anti-sickling β globin see, e.g., FIG. 16.

In some embodiments of the invention, a population of cells, which may be cells from a cell line or from an individual other than the subject, can be used. Methods of isolating stem cells, immune system cells, etc., from a subject and returning them to the subject are well known in the art. Such methods are used, e.g., for bone marrow transplant, peripheral blood stem cell transplant, etc., in patients undergoing chemotherapy.

Where stem cells are to be used, it will be recognized that such cells can be derived from a number of sources including bone marrow (BM), cord blood (CB) CB, mobilized peripheral blood stem cells (mPBSC), and the like. In certain embodiments the use of induced pluripotent stem cells (IPSCs) is contemplated. Methods of isolating hematopoietic stem cells (HSCs), transducing such cells and introducing them into a mammalian subject are well known to those of skill in the art.

In certain embodiments a Lenti-betaAS3-FB lentiviral vector is used in stem cell gene therapy for SCD by introducing the betaAS3 anti-sickling beta-globin gene into the bone marrow stem cells of patients with sickle cell disease followed by autologous transplantation. Such methods are illustrated herein in Example 1.

Direct Introduction of Vector.

In certain embodiments direct treatment of a subject by direct introduction of the vector is contemplated. The lentiviral compositions may be formulated for delivery by any available route including, but not limited to parenteral (e.g., intravenous), intradermal, subcutaneous, oral (e.g., inhalation), transdermal (topical), transmucosal, rectal, and vaginal. Commonly used routes of delivery include inhalation, parenteral, and transmucosal.

In various embodiments pharmaceutical compositions can include an LV in combination with a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

In some embodiments, active agents, i.e., a lentiviral described herein and/or other agents to be administered together the vector, are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such compositions will be apparent to those skilled in the art. Suitable materials can also be obtained commercially from Alza Corporation and Nova Pharmaceuticals, Inc. Liposomes can also be used as pharmaceutically acceptable carriers. These can be prepared according to methods known to those skilled in the art, for example, as described in U.S. Pat. No. 4,522,811. In some embodiments the composition is targeted to particular cell types or to cells that are infected by a virus. For example, compositions can be targeted using monoclonal antibodies to cell surface markers, e.g., endogenous markers or viral antigens expressed on the surface of infected cells.

It is advantageous to formulate compositions in dosage unit form for ease of administration and uniformity of dosage. Dosage unit form as used herein refers to physically discrete units suited as unitary dosages for the subject to be treated; each unit comprising a predetermined quantity of a LV calculated to produce the desired therapeutic effect in association with a pharmaceutical carrier.

A unit dose need not be administered as a single injection but may comprise continuous infusion over a set period of time. Unit dose of the LV described herein may conveniently be described in terms of transducing units (T.U.) of lentivector, as defined by titering the vector on a cell line such as HeLa or 293. In certain embodiments unit doses can range from 10³, 10⁴, 10⁵, 10⁶, 10⁷, 10⁸, 10⁹, 10¹⁰, 10¹¹, 10¹², 10¹³ T.U. and higher.

Pharmaceutical compositions can be administered at various intervals and over different periods of time as required, e.g., one time per week for between about 1 to about 10 weeks; between about 2 to about 8 weeks; between about 3 to about 7 weeks; about 4 weeks; about 5 weeks; about 6 weeks, etc. It may be necessary to administer the therapeutic composition on an indefinite basis. The skilled artisan will appreciate that certain factors can influence the dosage and timing required to effectively treat a subject, including but not limited to the severity of the disease or disorder, previous treatments, the general health and/or age of the subject, and other diseases present. Treatment of a subject with a LV can include a single treatment or, in many cases, can include a series of treatments.

Exemplary doses for administration of gene therapy vectors and methods for determining suitable doses are known in the art. It is furthermore understood that appropriate doses of a LV may depend upon the particular recipient and the mode of administration. The appropriate dose level for any particular subject may depend upon a variety of factors including the age, body weight, general health, gender, and diet of the subject, the time of administration, the route of administration, the rate: of excretion, other administered therapeutic agents, and the like.

In certain embodiments lentiviral gene therapy vectors can be delivered to a subject by, for example, intravenous injection, local administration, or by stereotactic injection (see, e.g., Chen et al. (1994) Proc. Natl. Acad. Sci. USA, 91: 3054). In certain embodiments vectors may be delivered orally or inhalationally and may be encapsulated or otherwise manipulated to protect them from degradation, enhance uptake into tissues or cells, etc. Pharmaceutical preparations can include a LV in an acceptable diluent, or can comprise a slow release matrix in which a LV is imbedded. Alternatively or additionally, where a vector can be produced intact from recombinant cells, as is the case for retroviral or lentiviral vectors as described herein, a pharmaceutical preparation can include one or more cells which produce vectors. Pharmaceutical compositions comprising a LV described herein can be included in a container, pack, or dispenser, optionally together with instructions for administration.

The foregoing compositions, methods and uses are intended to be illustrative and not limiting. Using the teachings provided herein other variations on the compositions, methods and uses will be readily available to one of skill in the art.

### EXAMPLES

The following examples are offered to illustrate, but not to limit the claimed invention.

### Example 1

### β-Globin Gene Transfer to Human Bone Marrow for Sickle Cell Disease

Autologous hematopoietic stem cell gene therapy is an approach to treating sickle cell disease (SCD) patients that may result in lower morbidity than allogeneic transplantation. We examined the potential of a LV (CCL-βAS3-FB) encoding a human beta-globin (HBB) gene engineered to impede sickle hemoglobin polymerization (HBBAS3) to transduce human BM CD34⁺ cells from SCD donors and prevent sickling of rbc produced by in vitro differentiation. The CCL-βAS3-FB LV transduced BM CD34⁺ cells from either healthy or SCD donors at similar levels, based on quantitative PCR and colony-forming unit progenitor analysis. Consistent expression of HBBAS3 mRNA and HbAS3 protein compromised a fourth of the total β-globin-like transcripts and Hb tetramers. Upon deoxygenation, a lower percentage of HBBAS3-transduced rbc exhibited sickling compared with mock-transduced cells from sickle donors. Transduced BM CD34⁺ cells were transplanted into immunodeficient mice, and the human cells recovered after 2-3 months were cultured for erythroid differentiation, which showed levels of HBBAS3 mRNA similar to those seen in the CD34⁺ cells that were directly differentiated in vitro. These results demonstrate that the CCL-βAS3-FB LV is capable of efficient transfer and consistent expression of an effective anti-sickling β-globin gene in human SCD BM CD34⁺ progenitor cells, improving physiologic parameters of the resulting rbc.

### Results

The CCL-βAS3-FB LV Vector Carrying the HBBAS3 Cassette.

The original LV produced by Levasseur et al. (19) to carry the HBBAS3 cassette (DL-βAS3) contained the intact HIV 5′ LTR, which engenders dependence on the HIV TAT protein for production of high-titer vector. To eliminate the need for TAT during packaging, we moved the HBBAS3 cassette plus the woodchuck hepatitis virus posttranscriptional regulatory element (WPRE) to the pCCL LV backbone (Dull et al. (1998) J. Virol. 72(11): 8463-8471), which is a SIN vector with the CMV enhancer/promoter substituted in the 5′ LTR, eliminating the need for TAT. This pCCL backbone was further modified to have a compact (77 bp) insulator in the U3 region of the 3′ LTR, denominated FB, which contains the minimal CTCF binding site (FII) of the 250-bp core of the 1.2-kb chicken β-globin HS4 (cHS4) insulator and the analogous region of the human T cell receptor 8/a BEAD-1 insulator (Ramezani et al. (2008) Stem Cells, 26(12): 3257-3266). The resulting SIN-LV was named CCL-βAS3-FB, and the proviral form is shown in FIG. 4A.

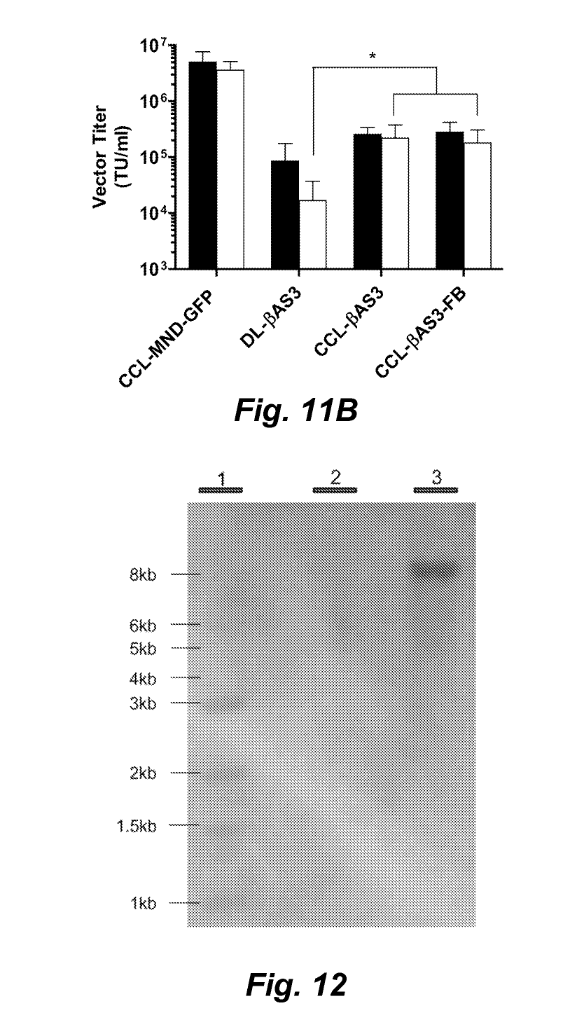

In three independent experiments, we packaged preparations of the CCL-βAS3-FB vector as well as a version lacking the FB insulator (CCL-βAS3), the parental DL-βAS3 vector, and a vector expressing the enhanced GFP (CCL-MND-GFP) as a positive control. The vector preparations were made with and without inclusion of a plasmid that expressed the HIV-1 TAT protein. The titers were determined by transducing a permissive cell line (HT29 human colon carcinoma) and measuring vector copies (VC)/cell using quantitative PCR (qPCR) with primers to the HIV packaging signal (Psi) of the vector proviruses (Sastry et al. (2002) Gene Ther. 9(17): 1155-1162; and FIGS. 11A and 11B). The CCL-βAS3-FB vector as well as the noninsulated version could be produced in the absence of TAT to a 10-fold higher titer than the original DL-βAS3 vector (P=0.017, 2-tailed t test; CCL-βAS3 and CCL-βAS3-FB combined compared with the DL-βAS3), and inclusion of the FB insulator did not decrease vector titer.

The stability of the FB insulator was evaluated by PCR analysis of the FB-containing fragment size in bulk populations of transduced BM CD34⁺ cells (FIG. 4B) and at a clonal level (a total of 32 single CFU colonies; data not shown). All samples showed the expected sizes of single bands after PCR analysis, demonstrating intact passage of the FB insulator. Additionally, Southern blot analysis of CCL-βAS3-FB-transduced cells showed the presence of a single band of the size expected for full-length vector provirus (FIG. 12).

To evaluate the functional activity of the FB insulator, binding of the CTCF protein to the LTRs of the CCL-βAS3-FB was assessed by ChIP in transduced K562 cells (FIG. 4C). ChIP indicated a 12-fold enrichment of CTCF binding in the CCL-βAS3-FB LTR when compared with the input control. No enrichment was found with the CCL-βAS3 vector lacking the FB insulator, indicating the specific binding of the CTCF to the FB sequence. The association with CTCF to the CCL-βAS3-FB LTR was at least as high as with other sequences known to bind CTCF, such as the 1.2-kb cHS4 insulator (Bell et al. (1999) Cell, 98(3): 387-396), the c-Myc promoter (Witcher and Emerson (2009) Mol. Cell. 34(3): 271-284), or the H19 imprinting control region (Bell and Felsenfeld (2000) Nature, 405(6785): 482-485).

Assessment of Transduction and Hematopoietic Potential of BM CD34⁺ Cells.

Preliminary dose-response experiments were performed to determine the most efficient concentration of the CCL-βAS3-FB vector to transduce human BM CD34⁺ cells, using a range of vector concentrations during transduction from 2×10⁶ to 2×10⁸ transduction units/ml (TU/ml) (MOI=4-400). A dose-related increase in gene transfer achieved (the average VC/cell measured by qPCR) was found only for vector concentrations below 2×10⁷ TU/ml. Higher vector concentrations did not increase the transduction efficacy and, in fact, often had a negative effect on the extent of transduction (data not shown). Based on these findings, the CCL-βAS3-FB vector was used at a standard concentration of 2×10⁷ TU/ml (MOI=40) for all subsequent studies.

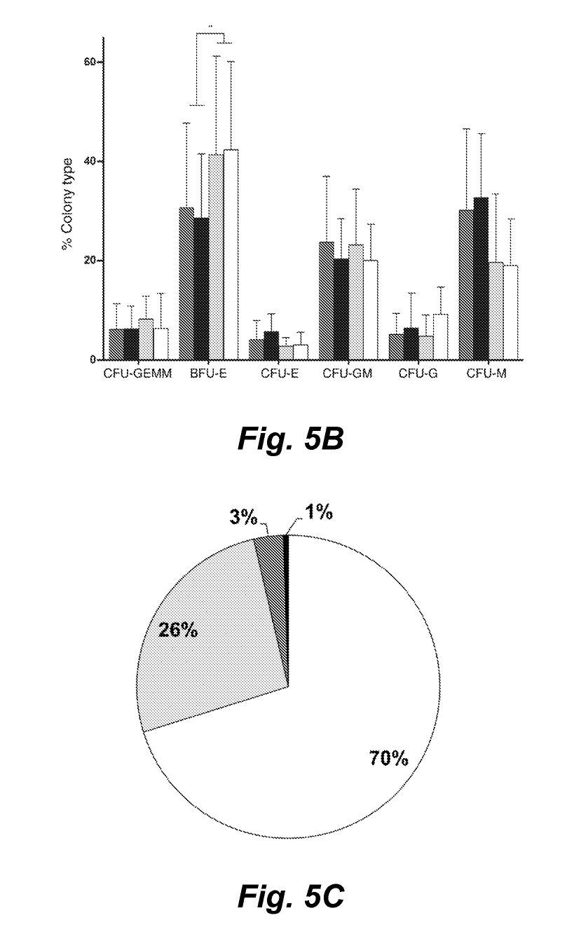

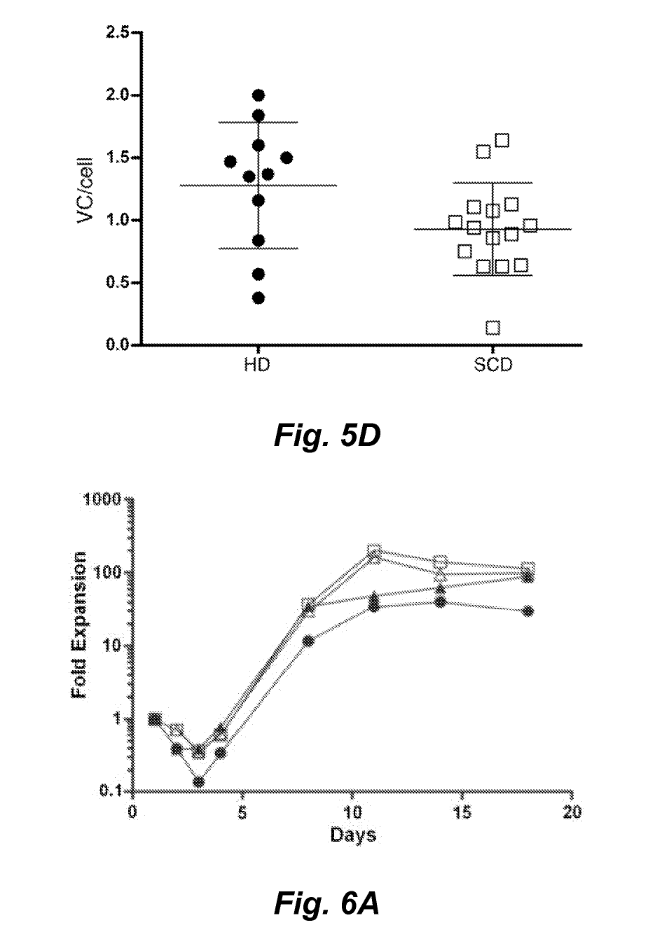

The colony-forming capacities of BM CD34⁺ cells were similar for samples from SCD donors or healthy donor (HD) controls, whether transduced with the CCL-βAS3-FB vector or not, with approximately 10% of cells forming colonies when plated in methylcellulose, without significant differences between groups (in all the groups compared, P>0.1, by 2-way ANOVA) (FIG. 5A). We noted higher percentages of burst-forming unit erythroid (BFU-E) (erythroid) colonies in SCD samples (41.34%±19.87% in SCD-mock and 42.33%±17.79% in SCD-βAS3-FB) compared with HD samples (30.67%±17.06% in HD-mock and 28.62%±12.91% in HD-βAS3-FB) (P=0.048, by 2-way ANOVA) (FIG. 5B). Similar erythroid skewing of progenitor cells from the BM of SCD patients has been reported (Croizat and Nagel R L (1988) Exp. Hematol. 16(11): 946-949) and may reflect the increased level of erythropoiesis in SCD patients due to the underlying hemolytic anemia. qPCR of individual CFU to detect the CCL-βAS3-FB vector sequences demonstrated the percentage of transduced colony forming progenitor cells from SCD donor BM. Fifty-seven of 191 colonies contained the CCL-βAS3-FB vector (29.84%±16.68% positive colonies in 5 independent experiments) with an average of 0.92±0.57 VC/cell in the bulk population cultured in vitro in erythroid differentiation conditions. Most of the vector-positive colonies analyzed had 1 to 2 VC/cell (88%), while 11% had 3 to 6 VC/cell and 2% had 7 to 9 VC/cell (no colony had more than 9 copies) (FIG. 5C). After 2 weeks of culture under in vitro erythroid differentiation conditions, transduction of CD34⁺ cells from HD (n=11) led to 1.28±0.51 VC/cell compared with 0.93±0.37 for SCD donors (n=15), which was borderline significantly different (P=0.05, Wilcoxon rank sum test) (FIG. 5D).

In Vitro Erythroid Differentiation of BM CD34⁺ Cells.

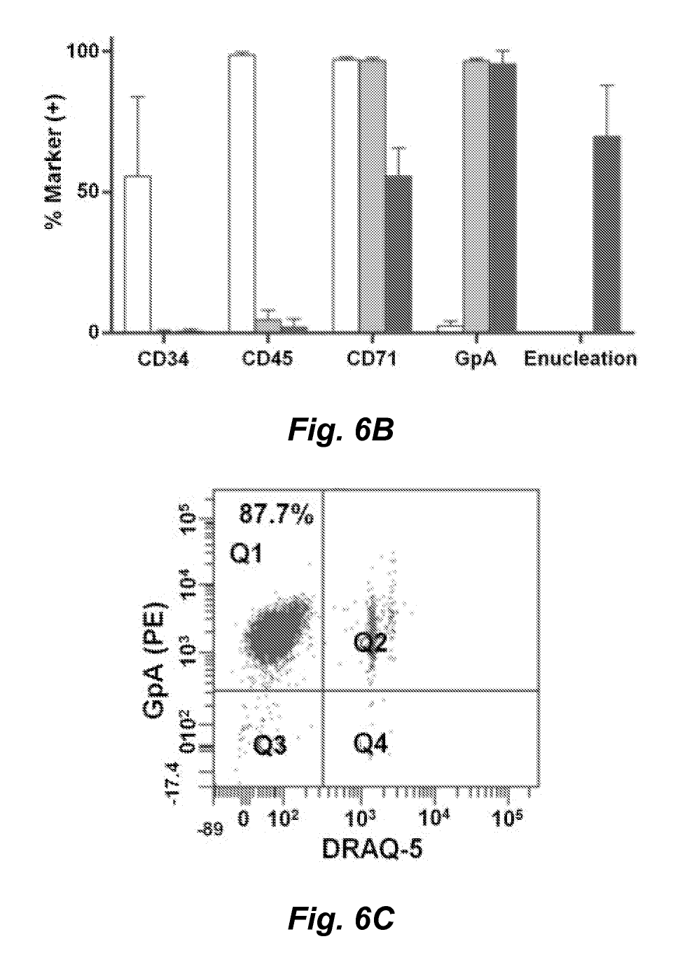

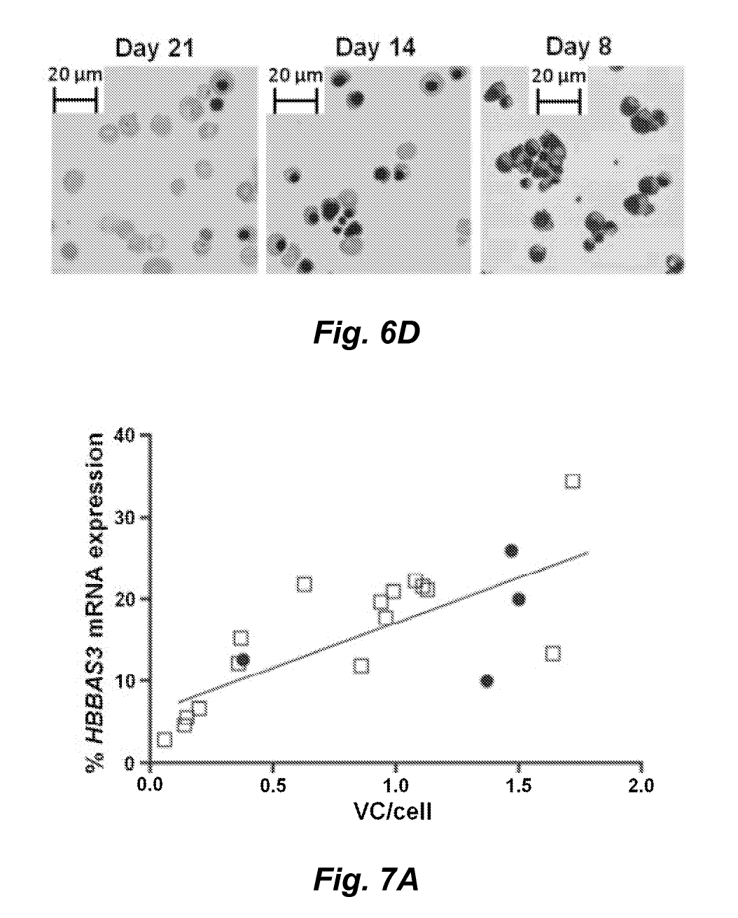

To assess expression of the erythroid specific HBB AS3 cassette, an in vitro model for supporting erythroid-directed differentiation from human BM CD34⁺ cells was used (Douay et al. (2009) Meth. Mol. Biol. 482: 127-140). CD34⁺ cells from the BM of SCD donors and HD were transduced with the CCL-βAS3-FB LV and control samples were mock-transduced. Starting 24 hours post transduction (pTD), the cells were differentiated for 21 days. During erythroid culture, the cells were counted serially over 3 weeks to determine viability and cell expansion. No differences in cell growth were found between HD and SCD donors for cells that were either transduced with the CCL-βAS3-FB LV or mock transduced (FIG. 6A shows a representative experiment). Expansion of cell numbers up to 700-fold was reached by the end of the culture. Flow cytometry was performed during erythroid differentiation culture to analyze the changes in markers of hematopoietic progenitors (CD34 and CD45) and erythroid progenitors (glycophorin A [GpA] and CD71). The percentage of CD34⁺ cells was analyzed after isolation, showing an average of 76.74%±3.01% of CD34⁺ cells. High variability in CD34 expression was observed after 3 days in culture between the different donors, with a sharp decline of CD34 expression between days 3 and 14 in all the samples (FIG. 6B). The pan-leukocyte marker CD45 was expressed by the entire cell population at day 3 and became essentially undetectable between days 14 and 21, as expected for reticulocytes and mature rbc (Migliaccio et al. (2002) Blood Cells Mol. Dis. 28(2): 169-180). CD71 (transferrin receptor) was expressed during the early part of the culture period (days 3 to 14), but decreased by the end of culture period as expected (day 21). GpA expression was detected on more than 90% of the cells by day 14 and persisted until the end of the culture.

Enucleated rbc were identified at the end of the differentiation (days 18 to 21) by double staining with an antibody to the erythroid membrane glycoprotein GpA and the fluorescent dye DRAQ5, which labels DNA; enucleated rbc were defined as being GpA+DRAQ5−. The frequency of enucleated rbc among multiple cultures ranged from 65% to 85%: 67.61%±17.68% in SCD-mock (n=7), 69.69%±18.11% in SCD-βAS3-FB (n=7) (FIG. 6B), 83.40%±10.07% in HD-mock (n=7) and 79.04%±10.19% in HD-βAS3-FB (n=3), without significant differences between mock-transduced and LV-transduced samples (SCD mock vs. βAS3-FB, P=0.80; HD mock vs. βAS3-FB, P=0.69, by 2-way ANOVA). The large-cell expansion and robust erythroid differentiation with high levels of enucleation (FIGS. 6C, and 6D) supported the further analyses to characterize the activity of the HBBAS3 transgene.

HBBAS3 mRNA Expression after In Vitro Erythroid Differentiation of BM CD34⁺ Cells.

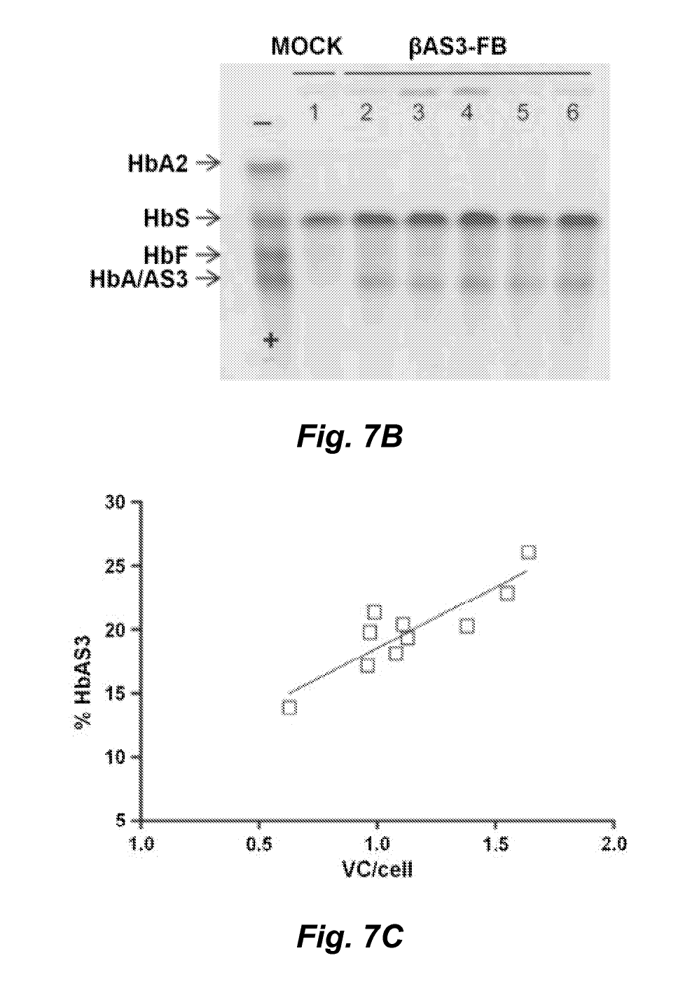

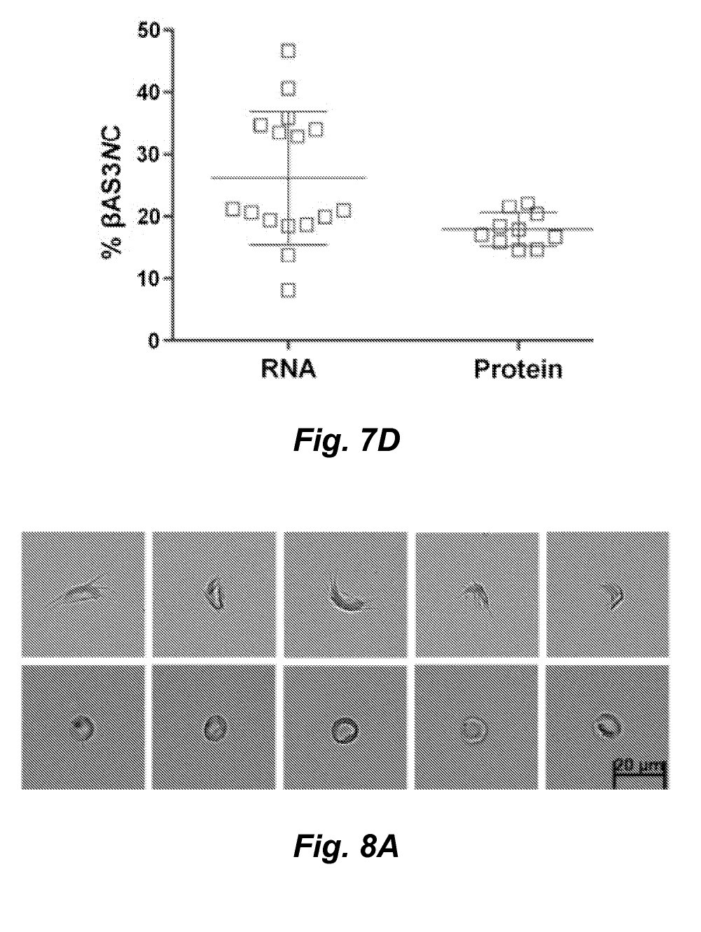

The successful production of rbc from BM CD34⁺ cells plus the confirmation of efficient gene transfer allowed us to evaluate the function of the HBBAS3 cassette. HBBAS3 mRNA expression levels in cells collected on day 14 from in vitro erythroid differentiation cultures of SCD donor and HD BM CD34⁺ cells, either transduced with the CCL-βAS3-FB LV or mock transduced, were assessed by a qRT-PCR assay and compared with mRNA levels from the endogenous HBB and HBBS (HBB gene carrying the sickle mutation) genes. HBBAS3 mRNA levels made up 15.73%±8.36% and 17.12%±7.25% of total β-globin-like mRNA in erythroid cells from cultures of SCD and HD BM CD34⁺ cells, respectively. For each CCL-βAS3-FB LV-transduced BM sample analyzed (SCD and HD), the percentage of HBBAS3 mRNA detected was compared with the VC/cell obtained by qPCR from that sample. There was a strong positive correlation between VC/cell and the percentage of HBBAS3 mRNA (Pearson correlation=0.73, P=0.0003), indicating consistent expression (FIG. 7A). When normalized to VC/cell to adjust for variable gene transfer, the average HBBAS3 mRNA expression per VC/cell, was 26.22%±10.71% in SCD and 17.84%±11.60% in HD cells. On average, from all the samples studied (n=20, 16 samples for SCD and 4 for HD) HBBAS3 mRNA comprised 24.55%±11.03% per VC/cell.

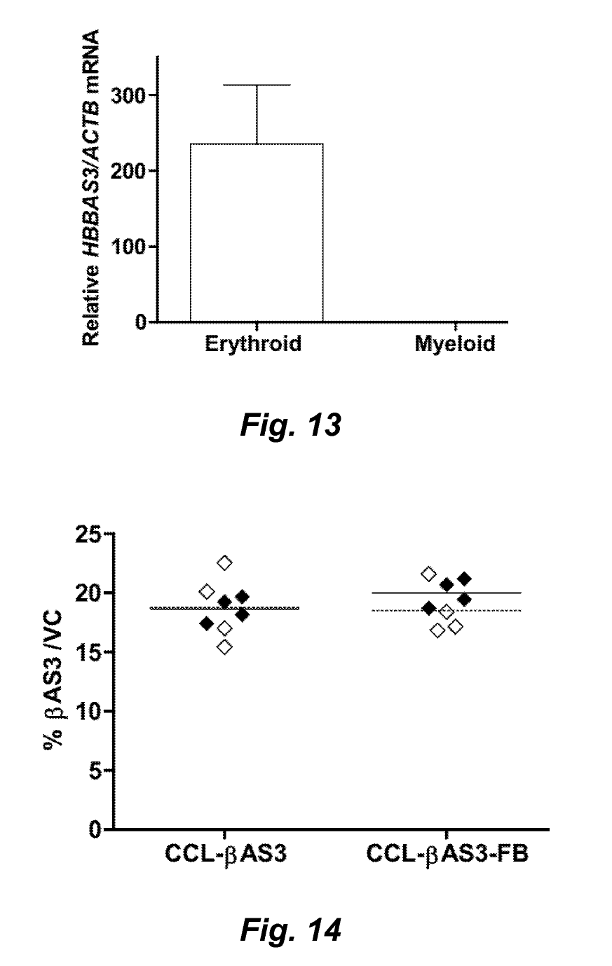

Finally, we assessed the erythroid specificity of expression of the HBBAS3 cassette by analyzing HBBAS3 mRNA expression in CCL-βAS3-FB LV-transduced BM CD34⁺ cells divided into parallel cultures under myeloid and erythroid differentiation conditions. We found a higher expression of HBBAS3 mRNA in cells produced under erythroid conditions compared with myeloid conditions, which was essentially unmeasurable (FIG. 13).

HbAS3 Protein Expression after In Vitro Erythroid-Differentiation of BM CD34⁺ Cells.

We used IEF to examine the Hb tetramers present in erythroid cells produced in vitro from BM CD34⁺ cells transduced with the CCL-βAS3-FB LV. Despite the 3 amino acid differences, HbAS3 tetramers cannot be distinguished from HbA by IEF because of their identical net charge. However, HbAS3 production can be readily distinguished from HbS, as the Glu6Val substitution introduced by the canonical sickle mutation deletes a negative charge in the protein, resulting in a more positive relative net charge of HbS. Therefore, only cells from SCD donors were analyzed for HbAS3 expression by IEF. An IEF membrane from a representative experiment is shown with 5 independent transductions of SCD BM CD34⁺ cells with the CCL-βAS3-FB LV, plus a mock-transduced sample (FIG. 7B).

In total, ten SCD samples were analyzed after erythroid differentiation. There was a strong correlation between the percentage of HbAS3 present in each sample and the extent of transduction measured by the VC (Pearson correlation=0.88, P=0.001) (FIG. 7C). A concomitant analysis of the some erythroid cell samples was performed by HPLC and IEF and showed similar results by both methods (Table 1).

TABLE 1
Measurement of % HbAS3 by HPLC and IEF

| | % HbAS3 | | | | | | |
|---|---|---|---|---|---|---|---|
| | or HBA | | % HbS | | % HbAS3/VC | | VC/ |
| | *HPLC | IEF | *HPLC | IEF | *HPLC | IEF | cell |
| SCD- | 0.00 | 0.00 | 100.00 | 100.00 | NA | NA | NA |
| MOCK #1 | | | | | | | |
| SCD- | 24.20 | 26.70 | 75.60 | 73.30 | 24.65 | 26.97 | 0.99 |
| βAS3FB #1 | | | | | | | |
| SCD- | 8.10 | 0.00 | 91.90 | 100.00 | NA | NA | NA |
| MOCK #2 | | | | | | | |
| SCD- | 15.20 | 9.50 | 84.40 | 90.50 | 15.78 | 21.11 | 0.45 |
| βAS3FB #2 | | | | | | | |

We then compared HBBAS3 RNA and protein expression levels normalized per VC/cell (FIG. 7D). While there was greater variability for HBBAS3 mRNA per VC/cell values compared with protein per VC/cell, the 2 methods indicated similar values of HBBAS3 expression (24.55%±11.03% HBBAS3 mRNA per VC/cell and 17.96%±3.09% HbAS3 protein per VC/cell), again indicating consistent expression. In four independent transductions, we compared the expression (mRNA and protein) from the HBBAS3 cassette in the presence or absence of the FB insulator (FIG. 14). We found that the addition of the FB insulator did not alter the expression of the HBBAS3 cassette when compared with the noninsulated LV.

SCD Phenotypic Correction.

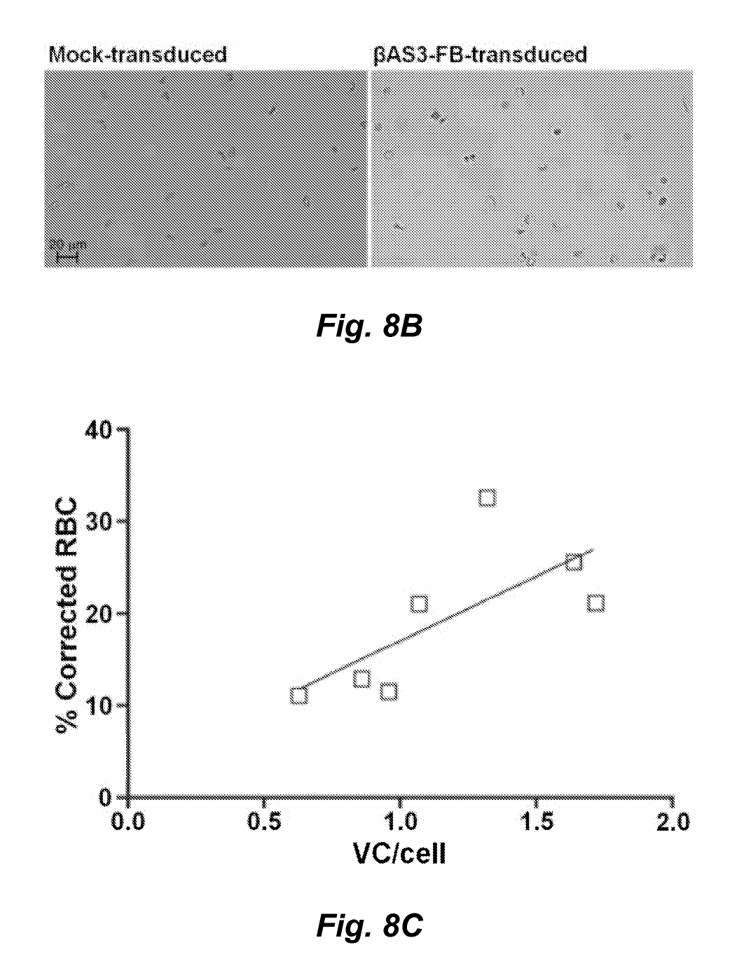

To assess the functional effects of HBBAS3 expression on the sickling of rbc produced in vitro from SCD BM CD34⁺ cells, we adapted and optimized an assay used in clinical laboratories to diagnose SCD: exposure of cells to the reducing agent sodium metabisulfite to induce HbS polymerization. rbc were harvested at the end of the erythroid culture (day 21) and incubated in sealed chambers of glass slides with sodium metabisulfite. After incubation, the morphology and shapes of the individual rbc were analyzed using phase-contrast microscopy to quantify the percentages of sickled-appearing rbc (srbc) and round, discoid nonsickled normal rbc (nrbc) (FIGS. 8A and 8B). In each experiment, 200-900 cells were analyzed for each sample. rbc from HD controls did not sickle in the presence of sodium metabisulfite, with more than 98% retaining their round morphology. In contrast, rbc produced in vitro from SCD BM CD34⁺ cells underwent sickling to a high extent in sodium metabisulfite, with averages of 88%±9% srbc and 12%±9% nrbc. In SCD samples transduced with the CCL-βAS3-FB LV, there was an increase in the percentage of rbc that did not undergo sickling, with 69%±16% srbc and 31%±16% nrbc, representing 19%±8% more nrbc compared with the nontransduced samples. These results demonstrated that expression by the CCL-βAS3-FB LV reduced rbc sickling during deoxygenation. The percentage of corrected sickle cells was positively correlated with the VC present (Spearman correlation=0.77, P=0.04) (FIG. 8C and Table 2).

TABLE 2
Enumeration of normal erythroid cells in SCD cells mock
transduced and transduced with the CCL-βAS3-FB LV.

| | | | | % nrbc | | % |
|---|---|---|---|---|---|---|
| Exp. | Donor Age | % | VC/ | | SCD- | Correc- |
| No | (yr) | HbF | cell | SCD-Mock | βAS3-FB | tion |
| 1 | 8 | 4.70 | 0.63 | 12.8 | 23.9 | 11.1 |
| 2 | 8 | 4.05 | 1.64 | 16.7 | 42.2 | 25.6 |
| 3 | 12, 8, 20^{A} | 0 | 0.96 | 4.8 | 16.4 | 11.5 |
| 4 | 12 | 0 | 0.86 | 1.6 | 14.6 | 12.9 |
| 5 | 12, 18, 21, | 0 | 1.72 | 3.7 | 24.8 | 21.2 |
| | 25, 27^{A} | | | | | |
| 6 | 27, 1^{A} | 5.40 | 1.07 | 18.7 | 39.8 | 21.1 |
| 7 | 12 | NA | 1.32 | 25.7 | 58.3 | 32.6 |

^{A}Multiple SCD-BM samples were pooled for these experiments.
NA, not analyzed.

In Vivo Assessment of CCL-βAS3-FB LV Transduction of BM CD34⁺ Cells.

To characterize the gene transfer and expression by the CCL-βAS3-FB LV in more primitive human hematopoietic stem and progenitor cells (HSPC), βAS3-FB-transduced BM CD34⁺ cells from SCD donors and HD controls were xeno-transplanted into immunodeficient NOD. Cg-Prkdc^{scid}Il2rg^{tm1Wjl}/SzJ (NSG) mice. Transduction conditions were the same as used for the in vitro analyses, and the cells were transplanted immediately after an overnight transduction. The transplanted cell doses ranged from 10⁵ to 10⁶ cells per mouse, depending on cell availability (BM source, cell dose, and number of mock- and βAS3-transduced mice used in each transplant are provided in Table 3). Eight to twelve weeks after transplant, the mice were euthanized and the BM was harvested for FACS analysis. Human cells recovered from the NSG BM were cultured under erythroid differentiation for further analysis.

TABLE 3
NSG mice transplant conditions.

| Transplant group | 1 | 2 | 3 | 4 | 5 | 6 |
|---|---|---|---|---|---|---|
| BM source | SCD | SCD | HD | HD | HD | HD |
| Cell dose | 9 × 10⁴ | 3 × 10⁵ | 10⁶ | 5 × 10⁵ | 10⁶ | 6.3 × 10⁵ |
| No. mock mice | 3 | 1 | 2 | 3 | 1 | 1 |
| No. βAS3-FB | 5 | 2 | 6 | 6 | 4 | 4 |
| mice | | | | | | |

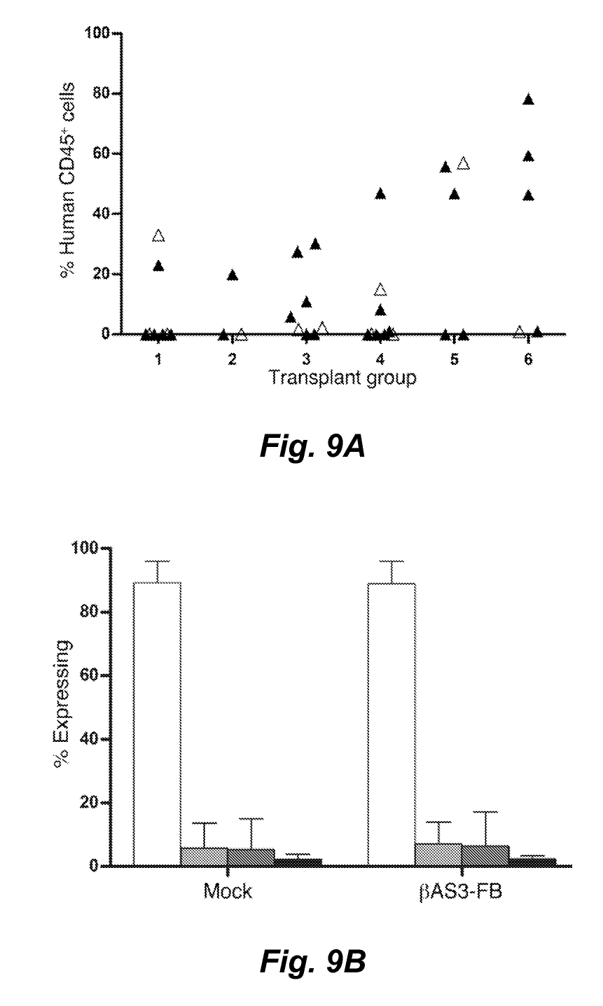

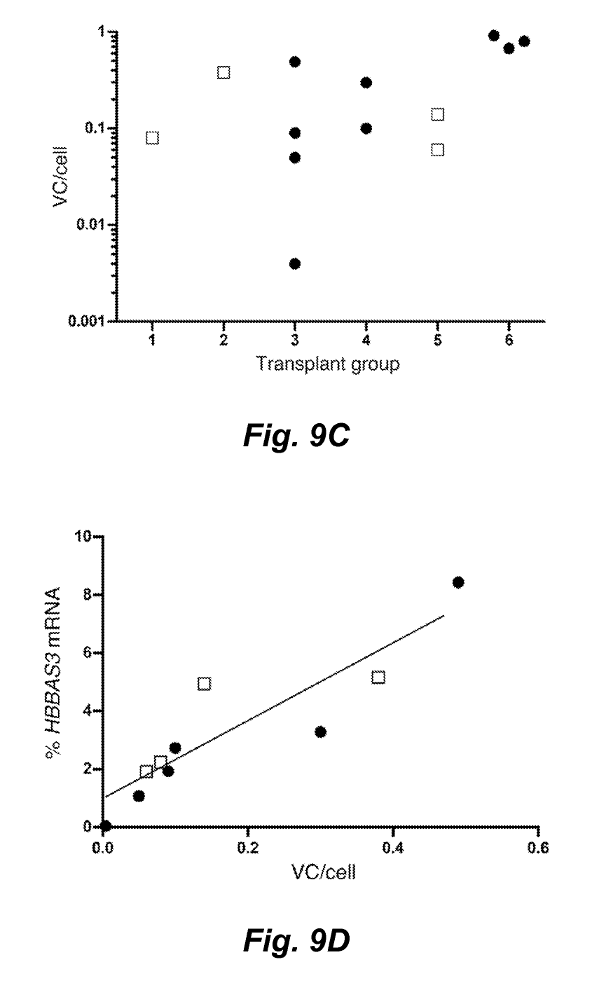

FACS analyses were performed to determine the engraftment of human cells in murine BM, defined as the percentage of human CD45⁺ cells of the total CD45⁺ population (murine CD45⁺ plus human CD45⁺). Engraftment values were variable among different transplants (up to 78%) (FIG. 9A). There were not consistent differences in engraftment using BM CD34⁺ cells from SCD donors or HD controls (P=0.6, by 2-way ANOVA) or between cells transduced with the βAS3-FB LV or mock-transduced (P=0.8, by 2-way ANOVA).

The human CD45⁺ populations from the transplanted mice were further analyzed for expression of markers for B-lymphoid cells (CD19), myeloid progenitors (CD33), hematopoietic progenitors (CD34), and erythroid cells (CD71). There were no differences in the relative proportions of the different types of human cells between mice engrafted with mock-transduced or CCL-βAS3-FB LV-transduced BM CD34⁺ cells, with the majority of human cells being B lymphoid cells (FIG. 9B), demonstrating that the transduction did not alter the differentiation potential of the cells.

BM was harvested from NSG mice, and human cells were enriched by depletion of murine CD45⁺ cells using immunomagnetic beads. The cells were then grown under in vitro erythroid differentiation conditions to induce terminal erythroid differentiation to allow the assessment of HBBAS3 mRNA expression by the CCL-βAS3-FB LV vector using qRT-PCR.

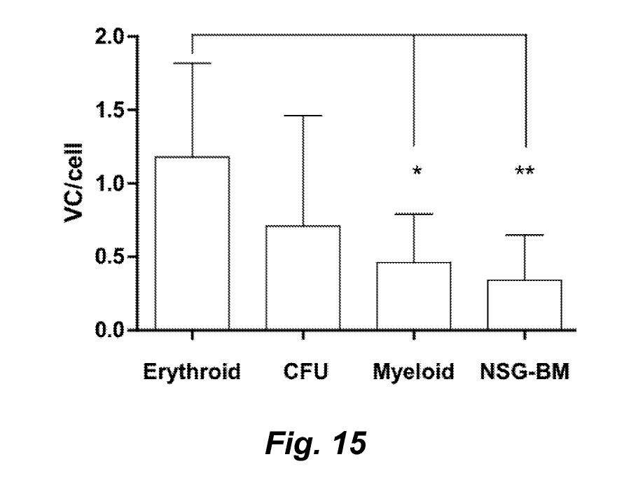

The VC/cell measured in cells grown from mice engrafted with human CD45+ cells ranged from 0.05 to 0.91 (FIG. 9C). Similar levels of gene marking were seen in samples from mice transplanted with BM CD34⁺ cells from SCD donors and HD (P=0.3, by 2-sample, 2-tailed t test). Overall, the VC/cell values assessed by qPCR were highest in cells grown in vitro under erythroid differentiation conditions (1.18±0.64 VC/cell), were lower in CFU (0.71±0.75 VC/cell) and cells produced by in vitro myeloid differentiation cultures (0.46±0.33 VC/cell), and were lowest in the human cells recovered from the NSG mice (0.34±0.31 VC/cell) (FIG. 15).

Quantification of HBBAS3 mRNA expressed by the human erythroid cells produced by in vitro erythroid differentiation of the cells isolated from the NSG mice was done using qRT-PCR. Expression of vector transcripts was correlated with VC/cells, with a mean value of 21.69%±8.35% of the total β-globin-like mRNA/VC (Pearson correlation=0.89, P=0.0004) (FIG. 9D). Thus, expression by the CCL-βAS3-FB LV was at a level in erythroid cells differentiated from the human cells engrafted in the NSG mice similar to that in transduced BM CD34⁺ cells that were directly differentiated in vitro. Genotoxicity assessment of the CCL-βAS3-FB LV. To evaluate the potential genotoxicity of the CCL-βAS3-FB LV, which contained strong erythroid enhancer elements as part of the lineage-specific β-globin expression cassette, two evaluations were performed: vector integration site (IS) analysis and an in vitro immortalization (IVIM) assay.

The vector IS in transduced human BM CD34⁺ cells were identified using nonrestrictive ligation-amplified PCR (nrLAMPCR) and mapping of the flanking sequences to the human genome with bioinformatic analyses. Comparisons were made between the patterns of the vector integration in the transduced BM CD34⁺ cells after a brief in vitro expansion versus after engraftment in NSG mice to look for evidence of preferential in vivo selection of clones containing integrants near cancer-associated genes (Higgins et al. (2007) Nucleic Acids Res. 35 (Database issue): D721-D726) or transcriptional start sites (TSS) as evidence of vector-related genotoxicity.

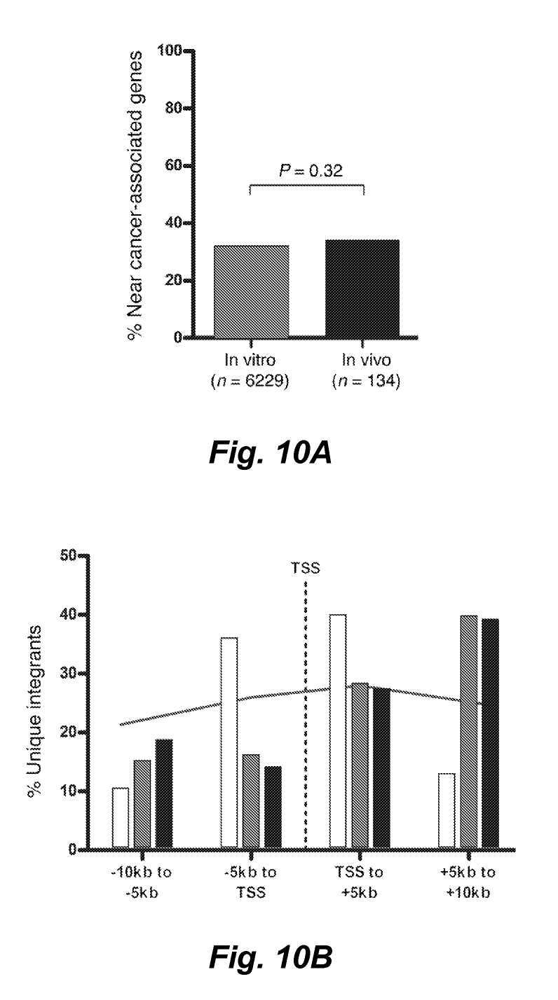

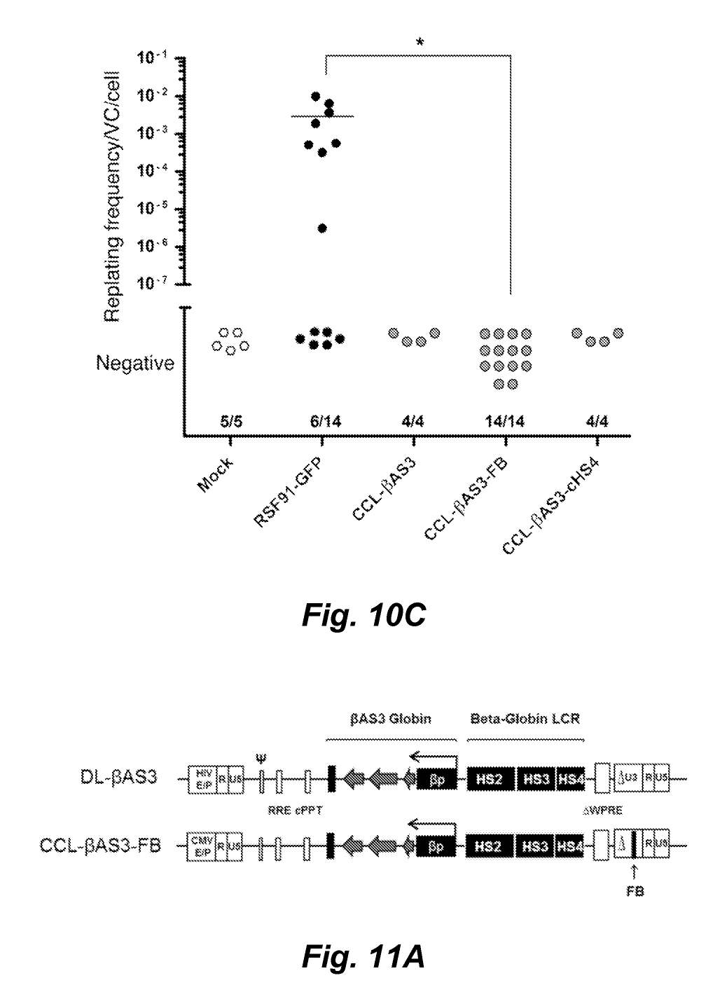

There were no increases in the percentages of vectors in proximity to cancer-associated genes following in vivo growth (binomial test, P=0.32; P value was determined using the binomial test, taking the proportion of cancer gene-proximal IS in the in vitro condition as an estimate of the probability of observing such an IS in engrafted mice) (FIG. 10A). There also was not an increased frequency of cells with vector integrations in proximity to TSS of genes (Table 4) compared with a random data set; in contrast, a comparative vector IS data set from a clinical trial using a γ-retroviral vector (Candotti et al. (2012) Blood, 1(18): 3635-3646) did show higher than random integrations near TSS (FIG. 10B).

TABLE 4
CCL-βAS3-FB most frequent integration
sites and the genes involved.

| | Orienta- | Nucleotide | Genes containing IS or with |
|---|---|---|---|
| | tion | Position | IS <50 kb from TSS |
| chr4 | + | 91503107 | FAM190A |
| chr16 | + | 1448144 | UNKL, C16orf42, GNPTG, C16orf91, |
| | | | CCDC154 |
| chr17 | − | 76027400 | TNRC6C |
| chr19 | + | 5631833 | SAFB, SAFB2, C19orf70, HSD11B1L |
| chr9 | + | 140097278 | LRRC26, MIR3621, ANAPC2, SSNA1, |
| | | | TPRN, TMEM203, NDOR1, |
| | | | RNF208, C9orf169, LOC643596, |
| | | | SLC34A3, TUBB2C, FAM166A, |
| | | | C9orf173 |
| chr22 | + | 24782983 | SPECC1L, ADORA2A |
| chr11 | − | 73279161 | FAM168A |
| chr6 | + | 34599566 | C6orf106 |
| chr16 | − | 20839013 | LOC81691, ERI2 |
| chr13 | + | 28784427 | PAN3 |
| chr17 | + | 29584884 | NF1, OMG |
| chr22 | + | 50820904 | PPP6R2 |
| chr22 | + | 38064948 | TRIOBP, SH3BP1, PDXP, LGALS1, |
| | | | NOL12 |
| chr12 | − | 62238951 | FAM19A2 |
| chr17 | + | 7158187 | DLG4, ACADVL, MIR324, DVL2, |
| | | | PHF23, GABARAP, CTDNEP1, |
| | | | C17orf81, CLDN7, SLC2A4, YBX2 |
| chr12 | + | 96696545 | CDK17 |
| chr5 | + | 88144268 | MEF2C |
| chr22 | + | 38784207 | LOC400927 |
| chrX | − | 153651194 | FLNA, EMD, RPL10, SNORA70, |
| | | | DNASE1L1, TAZ, ATP6AP1, GDI1, |
| | | | FAM50A, PLXNA3 |
| chr3 | + | 49120118 | USP19, QRICH1, QARS |
| chr19 | − | 6843325 | VAV1, EMR1 |
| chr4 | − | 7509127 | SORCS2, MIR4274 |
| chr5 | − | 77481360 | AP3B1 |
| chr16 | − | 1437062 | UNKL, C16orf42, GNPTG, C16orf91 |
| chr10 | + | 70679777 | DDX50, DDX21 |
| chr8 | − | 125342013 | TMEM65 |
| chr15 | − | 75352474 | PPCDC |
| chr11 | − | 96043251 | MAML2, MIR1260B |
| chr19 | − | 12287594 | ZNF20, ZNF625-ZNF20, ZNF625, |
| | | | ZNF136 |
| chr4 | + | 28371 | ZNF718, ZNF595 |
| chr9 | + | 75760126 | ANXA1 |
| chr1 | + | 31467152 | PUM1, SNORD103A, SNORD103B, |
| | | | SNORD85, PRO0611 |
| chr19 | + | 54072589 | ZNF331, LOC284379 |
| chr2 | + | 43510437 | THADA |
| chr9 | − | 140547986 | ARRDC1, EHMT1, C9orf37 |

To further assess the risk of insertional transformation by the βAS3-globin LV vectors, we performed genotoxicity studies using the IVIM assay that quantifies the immortalizing events by insertional transformation of murine lineage-negative BM cells grown in limiting dilution (Modlich et al. (2006) Blood, 108(8): 2545-2553). The immortalizing capacities of the LV vectors CCL-βAS3, CCL-βAS3-FB, and CCL-βAS3-cHS4 were compared with those of the γ-retroviral RSF91-GFP-wPRE as a positive control and with mock-transduced cells as a negative control. RSF91-GFP-wPRE carries the spleen focus-forming virus (SFFV) LTRs and is known to transform primary murine cells by insertional mutagenesis with a high probability in this assay.

Consistent with previous reports, the SFFV LTR-driven RSF91-GFP vector frequently generated clones (in 8 out of 14 transductions) with high replating frequencies of up to 5.26-02 (or 1 in 19 cells). In contrast, we found that in a total of 22 independent transductions (CCL-βAS3, n=4; CCL-βAS3-FB, n=14; and CCL-βAS3-cHS4, n=4), the βAS3-globin LV vectors did not give rise to any clones after the replating step (FIG. 10C and Table 5). In this in vitro setting, CCL-βAS3-FB was significantly less genotoxic than the SFFV LTR-driven γ-retroviral vector RSF91-GFP (P=0.002, by 2-sided Fisher's exact test) (FIG. 10C).

TABLE 5
IVIM assay results.

| | | | | | No. | No. | | |
|---|---|---|---|---|---|---|---|---|
| | | | | | positive | positive | | |
| | | | | | wells | wells | | |
| | Exp | Titer | | | (10² | (10³ | Replating | Replating |
| Vector | No | [TU/ml] | MOI | VCd.8 | cells/well) | cells/well) | frequency | frequency/VC |
| Non-transduced | 1 | — | — | — | 0 | 0 | — | — |
| | 2 | — | — | — | 0 | 0 | — | — |
| | 3 | — | — | — | 0 | 0 | — | — |
| | 4 | — | — | — | 0 | 0 | — | — |
| | 4 | — | — | — | 0 | 0 | — | — |
| RSF91-GFP | 1 | 1.9 × 10⁶ | 1 | 1.26 | 9 | 47 | 7.10E−04 | 5.63E−04 |
| | 1 | | 20 | 12.83 | 0 | 0 | — | — |
| | 2 | | 5 | 6.09 | 0 | 2 | 1.91E−05 | 3.14E−06 |
| | 2 | | 8 | 9.78 | 81 | 96 | 1.86E−02 | 1.90E−03 |
| | 3 | | 8 | 7.90 | 33 | 94 | 4.04E−03 | 5.12E−04 |
| | 3 | | 8 | 5.02 | 0 | 0 | — | — |
| | 4 | | 8 | 4.65 | 96 | 96 | >4.56E−02 | >9.81E−03 |
| | 4 | | 8 | 4.92 | 0 | 0 | — | — |
| | 4 | | 8 | 6.94 | 0 | 0 | — | — |
| | 4 | | 8 | 7.10 | 17 | 87 | 2.26E−03 | 3.18E−04 |
| | 4 | | 14 | 8.24 | 91 | 96 | 2.95E−02 | 3.59E−03 |
| | 4 | | 14 | 8.35 | 96, 96 | 96 | >5.26E−02 | >6.0E−03 |
| CCL-PAS3 | 1 | 1.5 × 10⁹ | 1000 | 1.02 | 0 | 0 | — | — |
| | 2 | | 100 | 1.10 | 0 | 0 | — | — |
| | 3 | 5.0 × 10⁹ | 100 | 2.32 | 0 | 0 | — | — |
| | 3 | | 100 | 3.39 | 0 | 0 | — | — |
| CCL-PAS3-FB | 1 | 6.0 × 10⁸ | 1000 | 4.68 | 0 | 0 | — | — |
| | 1 | | 100 | 4.76 | 0 | 0 | — | — |
| | 2 | | 100 | 4.32 | 0 | 0 | — | — |
| | 2 | | 100 | 4.52 | 0 | 0 | — | — |
| | 3 | 6.0 × 10⁸ | 100 | 3.66 | 0 | 0 | — | — |
| | 3 | | 100 | 3.31 | 0 | 0 | — | — |
| | 4 | | 100 | 3.22 | 0 | 0 | — | — |
| | 4 | | 100 | 3.56 | 0 | 0 | — | — |
| | 4 | | 100 | 3.23 | 0 | 0 | — | — |
| | 4 | | 100 | 4.23 | 0 | 0 | — | — |
| | 4 | | 100 | 4.55 | 0 | 0 | — | — |
| | 4 | | 100 | 3.49 | 0 | 0 | — | — |
| | 4 | | 100 | 3.49 | 0 | 0 | — | — |
| | 4 | | 100 | 2.56 | 0 | 0 | — | — |
| CCL-pAS3-cHS4 | 2 | 1.6 × 10⁸ | 100 | 0.30 | 0 | 0 | — | — |
| | 3 | | 100 | 0.54 | 0 | 0 | — | — |
| | 3 | | 100 | 0.36 | 0 | 0 | — | — |
| | 3 | | 100 | 0.41 | 0 | 0 | — | — |

### DISCUSSION

We performed studies using human BM CD34⁺ cells from SCD donors to assess the potential suitability of the CCL-βAS3-FB LV to achieve the requisite levels of transfer and expression of the anti-sickling HBBAS3 gene to inhibit sickling in rbc. BM is the likely autologous HSC source that would be used clinically for gene therapy in SCD because of the increased risks from mobilization of PBSC with G-CSF in SCD patients (Abboud et al. (1998) Lancet 351(9107): 959; Adler et al. (2001) Blood, 97(10): 3313-3314; Fitzhugh et al. (2009) Cytotherapy, 11(4): 464-471).

In allogeneic HSCT for SCD, stable donor HSC chimerism of 10%-30% can lead to significant hematologic and clinical improvement due to a selective survival advantage of the normal donor-derived rbc compared with the shortened survival of the HbS-containing recipient-derived rbc (Walters et al. (2001) Biol. Blood Marrow Transplant. 7(12): 665-673; Andreani et al. (2010) Haematologica, 96(1): 128-133; Wu et al. (2007) Br. J. Haematol. 139(3): 504-507; Krishnamurti et al. (2008) Biol. Blood Marrow Transplant. 14(11): 1270-1278). In SCD patients with HPFH, levels of HbF of 8%-15% or more (Platt et al. (1994) N. Engl. J. Med. 330(23): 1639-1644; Charache et al. (1987) Blood, 69(1): 109-116) ameliorate the severity and frequency of clinical symptoms. These clinical findings define the minimum threshold for autologous transplant of gene-corrected HSC to benefit SCD because it is unknown whether rbc expressing the HBBAS3 gene will be as beneficial as rbc expressing only HBB from an HD. Hence, at least 10%-30% engrafted gene-corrected HSC producing rbc expressing at least 8%-15% HbAS3 would be needed to potentially achieve the same therapeutic effect as a similar level of allogeneic donor engraftment. Human CD34⁺ cells are relatively resistant to gene transfer by LV vectors compared with permissive cell lines, and this is accentuated when the vector titers are low. Thus, a key challenge is transducing a sufficient percentage of the CD34⁺ cells to lead to engraftment of gene-corrected HSC at the needed frequencies (e.g., 10%-30%). Stable engraftment of 10%-20% gene-modified autologous HSC has been demonstrated in clinical trials for X-ALD and β-thalassemia using LV vectors and fully cytoablative conditioning, indicating that it should be achievable in the setting of SCD as well (Cavazzana-Calvo et al. (2010) Nature, 467(7313): 318-322; Cartier et al. (2009) Science 326(5954): 818-823). In our study, the CFU assay demonstrated that 30% of the colony-forming progenitors were transduced. It is believed that transduction of this percentage of engrafting HSC is within the target range for a clinical trial.

The anti-sickling activity of the HBBAS3 gene was shown to be equivalent to HbF in vitro (Levasseur et al. (2004) J. Biol. Chem. 279(26): 27518-27524) so production of HbAS3 at greater than 8%-15% of total Hb levels may inhibit sickling in a clinically beneficial manner. In a murine model of SCD, the parental LV DL-βAS3 expressed HbAS3 at 20%-25% of the total Hb, with the remainder coming from the human HBBS transgene (Levasseur et al. (2003) Blood, 102(13):4312-4319). These prior results suggest that LV-mediated transfer of the HBBAS3 gene could be clinically efficacious in gene therapy. In our study, the expression and functional activity of the CCL-βAS3-FB LV was remarkably consistent and effective. There was a very reproducible level of expression of the HBBAS3 gene by the vector in primary human erythroid cells produced from transduced BM CD34⁺ cells, making up 15%-25% of the total β-globin-like mRNA transcripts and Hb tetramers. Expression of the HbAS3 protein consistently increased the percentage of rbc produced from CCL-βAS3-FB-transduced SCD CD34⁺ cells that did not sickle upon deoxygenation, indicating a functional protection similar to the effect of γ-globin expression. These results are consistent with the initial studies with the HBBAS3 gene by Townes and colleagues, in which the parental DL-βAS3 LV corrected abnormal rbc morphology and hematologic parameters in BM-transplanted SCD mice (Levasseur et al. (2003) Blood, 102(13):4312-4319).

We have achieved vector transduction levels and HbAS3 protein production within the target range. However, a higher percentage of HSC bearing the HBBAS3 transgene would likely provide a larger population of rbc containing the anti-sickling HbAS3 and therefore may provide greater clinical benefit. Attempts to improve β-globin LV vectors have shown that removing β-globin regulatory elements increased titer and transduction efficiency; however, this compromised expression levels (Lisowski and Sadelain (2007) Blood, 110(13): 4175-4178). Further efforts to improve the transduction efficiency of β-globin vectors without compromising their transgene expression would be an important advance in the field. We developed and tested a derivative of the original DL-βAS3 LV (Levasseur et al. (2003) Blood, 102(13):4312-4319), named CCL-βAS3-FB, replacing the HIV promoter in the 5′ LTR with the CMV enhancer/promoter to eliminate the need for expressing the HIV TAT protein during the packing process (Dull et al. (1998) J. Virol. 72(11): 8463-8471). This modification in the original LV backbone may improve the biosafety of the vector by eliminating the TAT gene from the packaging step. It also led to at least a 10-fold increase of the vector titers when compared with the original. However, despite this improvement, the large amount of regulatory elements needed for high-level expression of the β-globin gene makes this type of LV complex and lowers the achievable titers when compared with vectors with simpler gene cassettes.

In some gene therapy settings in which strong enhancers and other regulatory elements are needed for sufficient expression of a transferred gene (e.g., chronic granulomatous disease, β-thalassemia), the genotoxic potential of these elements may be diminished when insulator elements are added (Emery et al. (2000) Proc. Natl. Acad. Sci., USA, 97(16): 9150-9155). Insulators are DNA sequences that act as boundary elements to inhibit interactions between adjacent chromatin domains, which can manifest as either enhancer-blocking activity, heterochromatin barrier activity, or both. The enhancer-blocking activity of insulators would reduce trans-activation of transcription from promoters of adjacent cellular genes. The barrier activity of insulators would decrease transgene silencing caused by spreading of surrounding heterochromatin into the vector provirus (Raab and Kamakaka (2010) Nat. Rev. Genet. 11(6): 439-446).

The major DNA-binding protein associated with enhancer blocking activity of insulators in vertebrates is the CTCF (CCCTC binding factor) protein (Bell et al. (1999) Cell, 98(3): 387-396), a highly conserved and ubiquitous zinc finger protein (Lobanenkov et al. (1990) Oncogene, 5(12): 1743-1753; Filippova et al. (1996) Mol. Cell Biol. 16(6): 2802-2813; Vostrov and Quitschke (1997) J. Biol. Chem. 272(52): 33353-33359). The FB insulator used in the CCL-βAS3-FB LV was previously shown to have enhancer-blocking activity similar to the full 1.2-kb cHS4 insulator in a reporter plasmid transfection assay and exceeding that of the 250-bp core cHS4 insulator fragment (Ramezani et al. (2008) Stem Cells, 26(12): 3257-3266).

In the CCL-βAS3-FB LV, the relatively small FB insulator (77 bp) did not lower the titers of the parental CCL-βAS3 LV when inserted into the U3 region of the 3′ LTR. It was transmitted faithfully to the 5′ LTR during RT, with no detectable deletion or losses in the vector provirus by Southern blot analysis or by PCR analysis of the FB insulator region from pools of transduced human CD34⁺ cells and from clonal CFUs grown in vitro. We could not assess the functional ability of the FB insulator to decrease risks for genotoxicity in the IVIM assay because neither the parental vector lacking the FB insulator nor the CCL-βAS3-FB LV caused any clonal outgrowth. However, we did observe evidence of in vitro activity of the FB insulator based on the greatly enriched binding of CTCF protein to LTR regions of the CCL-βAS3-FB, as assessed by ChIP analysis from K562 cells.

In light of the recent report of aberrant splicing into the 250-bp cHS4 insulator element in an LV vector used for transduction of BM CD34⁺ cells in a trial for β-thalassemia (Cavazzana-Calvo et al. (2010) Nature, 467(7313): 318-322), we performed an in silico splice site analysis of the FB insulator sequences. Whereas the NetGene2 server (Brunak et al. (1991) J. Mol. Biol. 220(1): 49-65) identified the cryptic splicing site seen in the cHS4 insulator by Cavazzana-Calvo et al. (2010) Nature, 467(7313): 318-322, it did not predict splicing signals in an FB-containing SIN LTR. These studies indicate that the FB insulator does not lower vector titers, is transmitted intact, binds the major cellular factor responsible for producing enhancer-blocking activity, and is not predicted to serve as a cryptic splice site; however, it is unknown whether the presence of the FB insulator in the vector will increase safety in clinical applications.

Safety assessments using the IVIM assay with CCL-βAS3-FB-transduced murine BM cells and vector IS analyses of human BM CD34⁺ cells transplanted in vivo to NSG mice did not reveal any evidence of genotoxicity, although the sensitivity of these surrogate assays may be relatively low. The observed pattern of vector IS for the LV was consistent with those described previously for HIV-1-based LV, with preferential integration into genes and no preference for integrations near TSS (Wu et al. (2003) Science 300(5626): 1749-1751). This contrasted with a recently published γ-retroviral IS data set (Candotti et al. (2012) Blood, 1(18): 3635-3646).

In all, these studies provide preclinical data for sufficiently effective transduction of human BM CD34⁺ progenitor from SCD patients to support translation to a clinical trial of gene therapy for SCD using the CCL-βAS3-FB LV. Outcomes from autologous transplants of gene-modified HSC will need to be compared with those from allogeneic transplant approaches, which continue to advance, to define the clinical utility of gene therapy for SCD.

### Methods

BM CD34⁺ Cell LV Transduction.

For transduction, BM CD34⁺ cell samples from SCD and HD were thawed and plated at 1×10⁶ cells/ml in tissue culture plates precoated with RetroNectin (20 μg/ml, Takara Shuzo Co.). Prestimulation was performed for 18-24 hours in X-Vivo 15 medium (Lonza) containing 1× glutamine, penicillin, and streptomycin (Gemini Bio-Products). Cytokines were added at the following concentrations: 50 ng/ml human SCF (hSCF) (StemGent), 50 ng/ml human hFlt3 ligand (hFlt3-1) (PeproTech), 50 ng/ml human thrombopoietin (hTPO), and 20 ng/ml human IL-3 (hIL-3) (both from R&D Systems). Cells were transduced with concentrated viral supernatants of the CCL-βAS3-FB LV at a final concentration of 2×10⁷ TU/ml (MOI=40, based on titers on HT29 cells) for all experiments done. Twenty-four hours after transduction, the cells were plated in methylcellulose for CFU assay and were also plated in in vitro erythroid differentiation culture and used for xeno-transplant into NSG mice.

In Vitro Erythroid Differentiation Culture.

The in vitro erythroid differentiation technique used is based on a 3-phase protocol adapted from Giarratana et al. (Douay et al. (2009) Meth. Mol. Biol. 482: 127-140). After 2 days of culture, for prestimulation and transduction, cells were transferred into erythroid culture. The basic erythroid medium was Iscove's Modified Dulbecco's Medium (IMDM; Life Technologies) (lx glutamine, penicillin, and streptomycin) supplemented with 10% BSA, 40 μg/ml inositol, 10 μg/ml folic acid, 1.6 μM monothioglycerol, 120 μg/ml transferrin, and 10 μg/ml insulin (all from Sigma-Aldrich). During the first phase (6 days), the cells were cultured in the presence of 10-6 M hydrocortisone (Sigma-Aldrich), 100 ng/ml hSCF, 5 ng/ml hIL-3, and 3 IU/ml erythropoietin (Epo) (Janssen Pharmaceuticals). In the second phase (3 days), the cells were transferred onto a stromal cell layer (MS-5, murine stromal cell line (Suzuki et al. (1992) Leukemia, 6(5): 452-458) (provided by Gay Crooks, UCLA) with the addition of only Epo (3 IU/ml) to basic erythroid medium. At day 11, all the cytokines were removed from the medium and the cells were cocultured on the MS-5 stromal layer until days 18 to 21.

qPCR for Determination of VC/Cell.

On day 14 of the erythroid differentiation, 10⁵ cells from the erythroid cultures were harvested for genomic DNA isolation using the PureLink Genomic DNA Mini Kit (Invitrogen). The average VC/cell was determined by multiplex qPCR of the HIV-1 packaging signal sequence (Psi) in the LV provirus and normalized to the cellular autosomal gene syndecan 4 (SDC4) to calculate the average VC/cell. This multiplex qPCR method was previously described (Cooper et al. (2011) J. Virol. Meth. 177(1): 1-9).

HBBAS3 mRNA Quantification by qRT-PCR.

To determine HBBAS3 mRNA expression, 1 to 2×10⁵ cells were harvested on day 14 of erythroid differentiation. RNA was extracted using the RNeasy Plus Mini Kit (QIAGEN) according to the manufacturer's instructions. The genomic DNA elimination columns contained in the kit were used to eliminate possible DNA contamination during the extraction. First-strand cDNA was synthesized using random primers, M-MLV reverse transcriptase, and RNAseOUT Recombinant Ribonuclease Inhibitor (all from Invitrogen) according to the manufacturer's protocol. SYBR Green qPCR amplification of cDNAs was performed using Platinum Taq DNA Polymerase (Platinum SYBR Green qPCR SuperMix; Invitrogen) on a ViiA7 Real-Time PCR System (Applied Biosystems).

To specifically detect mRNA transcripts originating from the vector CCL-βAS3-FB (HBBAS3 mRNA) in differentiated rbc and compare them with the levels of endogenous β-globin-like mRNA (HBB in HD samples and HBBS in SCD samples, respectively), 2 sets of allele-specific primers were designed (HBB^{A}/HBB^{S} and HBB^{AS3}; Table 6). The percentage of HBBAS3 transcripts (% HBBAS3) among all β-globin-like transcripts was determined from the relative expression of HBBAS3 vs. HBB and HBBS transcripts, respectively, comparing absolute numbers of transcripts per μl cDNA measured using an absolute plasmid standard curve ranging from 10⁸ to 10¹ molecules/μl DNA. Both primer sets were used in a 2-step PCR protocol with the denaturation step at 95° C. for 15 seconds and the annealing/extension step at 72° C. for 1 minute for a total of 40 cycles. All reactions were performed in duplicate, and dissociation curve analysis was carried out for each reaction to rule out nonspecific amplification.

HbAS3 Tetramer Quantification by IEF.

Hb IEF was performed using the Hemoglobin Electrophoresis Procedure (Helena Laboratories) according to the manufacturer's instructions. Briefly, a minimum of 3×10⁶ cells were harvested on day 21 of erythroid differentiation. The cells were lysed with Hemolysate Reagent (Helena Laboratories) as per instructions and incubated overnight at 4° C. If necessary, lysates were concentrated the next day using Micron Centrifugal Filters (Ultracel YM-30; Millipore); 5 μl of the samples were loaded onto a Titan III cellulose acetate plate (Helena Laboratories) and electrophoresed for 25 minutes at 350 volts. The plate was stained by Ponceau S (Sigma-Aldrich) for visualization of the Hb tetramers, cleared using Clear Aid solution (Helena Laboratories), and dried. The Hb bands were identified by comparison with Helena Hemo Controls and quantified by densitometry using ImageQuant TL software (GE Healthcare).

SCD Phenotypic Correction Assay.

At day 21 of the erythroid differentiation, 2.5×10⁵ cells per condition were harvested for SCD phenotypic correction assay. The samples were spun down (500 g for 5 minutes), and the resulting pellets were harvested in 10 μl of the supernatant; 10 μl of 20 μg/ml Sodium Metabisulfite (Sigma-Aldrich) was added to each sample. This mix was loaded onto a glass microscope slide, covered, and sealed at the edges. The samples were incubated at 5% CO2, 37° C. for 25-40 minutes. Images of the cells were then captured by inverse microscopy with a Nikon DS-Fi1 camera, from consecutive fields at ×10 magnification. Computer vision was utilized to isolate cells within each field and then individually present them to the user for visual analysis of normal or sickle morphology in a randomized and unbiased fashion across treatment groups.

Transplantation of Transduced Human BM CD34⁺ Cells in Immunodeficient Mice.

BM CD34⁺ cells from HD or SCD donors transduced with the CCL-βAS3-FB vector or mock transduced (10⁵-10⁶ cells) were transplanted by tail-vein injection into 9- to 12-week-old, NSG mice (The Jackson Laboratory) after 250 cGy total body irradiation. After 8-12 weeks, mice were euthanized and BM was analyzed for engraftment of human cells by flow cytometry using APC-conjugated anti-human CD45 vs. FITC-conjugated anti-murine CD45. After antibody incubation, rbc were lysed using BD FACS-Lysing Solution (BD Biosciences). The percentage of engrafted human cells was defined as follows: % huCD45⁺/(% huCD45⁺+% muCD45⁺). Analysis of the different hematopoietic cell types present was performed by staining for peridinin-chlorophyll-conjugated (PerCP) anti-human CD34, V450-conjugated anti-human CD45, FITC-conjugated anti-human CD19, PE-conjugated anti-human CD33, and APC-conjugated anti-human CD71 (all antibodies from BD Biosciences). BM from engrafted mice was depleted of murine CD45′ cells using immunomagnetic separation (CD45 MicroBeads-mouse; Miltenyi Biotech, Bergisch Gladbach). The mCD45-negative fraction was cultured for in vitro erythroid differentiation as described above to produce cells for analysis of the VC/cell and HBBAS3 mRNA expression. For each sample, qPCR was performed using primers to amplify the packaging (Psi) region of the provirus and normalized for DNA copy using primers to the autosomal human gene SDC4 (Cooper et al. (2011) J. Virol. Meth. 177(1): 1-9) to adjust for the potential presence of murine cells in the cultures.

Vector IS Analysis.

Depending on availability, 1-100 ng of genomic DNA isolated from cells were used to perform nonrestrictive linear amplification-mediated (nr-LAM) PCR to identify vector IS (Paruzynski et al. (20100 Nat. Protoc. 5(8): 1379-1395). Briefly, 100 cycles of linear amplification were performed with primer HIV3 linear (biotin-AGT AGT GTG TGC CCG TCT GT (SEQ ID NO:1)). Linear reactions were purified using 1.5 volumes of AMPure XP beads (Beckman Genomics) and captured onto M-280 Streptavidin Dynabeads (Invitrogen Dynal). Captured ssDNA was ligated to read 2 linker (phos-AGA TCG GAA GAG CAC ACG TCT GAA CTC CAG TCA C-3C spacer (SEQ ID NO:2)) using CircLigase II (Epicentre) in a 10-μl reaction at 65° for 2 hours. PCR was performed on these beads using primer HIV3 right (AAT GAT ACG GCG ACC ACC GAG ATC TAC ACT GAT CCC TCA GAC CCT TTT AGT C (SEQ ID NO:3)) and an appropriate indexed reverse primer (CAA GCA GAA GAC GGC ATA CGA GAT-index-GTG ACT GGA GTT CAG ACG TGT (SEQ ID NO:4)). PCR products were mixed and quantified by probe-based qPCR, and appropriate amounts were used to load Illumina v3 flow cells. Paired-end 50-bp sequencing was performed on an Illumina HiSeq 2000 instrument using a custom read 1 primer (CCC TCA GAC CCT TTT AGT CAG TGT GGA AAA TCT CTA GCA (SEQ ID NO:5)).

Reads were aligned to the hg19 build of the human genome with Bowtie (Langmead et al. (2009) Genome Biol. 10(3): R25), and alignments were condensed and annotated using custom Perl and Python scripts to locate vector integrations relative to RefSeq gene annotations obtained from the UCSC database. The frequencies of IS in transcribed regions of or within 50 kb of promoters of cancer-associated genes (as defined in Higgins et al. (2007) Nucleic Acids Res. 35 (Database issue): D721-D726) were determined. See Supplemental Methods below for details of LV vector construction, production and titration, PCR for FB insulator integrity, Southern blot, ChIP, BM CD34⁺ cell isolation, CFU progenitor assay, myeloid culture, flow cytometry during erythroid culture, IVIM assay, and HBBAS3 mRNA expression in erythroid and myeloid conditions.

Statistics.

Descriptive statistics of continuous outcome variables such as the mean and SD by experimental conditions are presented in figures. For continuous outcomes such as titer, VC/cell, percentage of enucleation, percentage of colonies grown, etc., 1-way or 2-way ANOVA (Tukey (1957) Ann. Math Statist. 28(1): 43-56) was used to assess overall group difference, depending on the experimental designs. Further, we performed 2-group comparison by 2-sample t test (within the framework of ANOVA if more than 2 groups) or Wilcoxon rank sum test if normality assumption was not met. Pearson correlation (Snedecor and Cochran W G (1989) Statistical Methods. 7th ed. Ames, Iowa, USA: Iowa State University Press) was used to measure the correlation between VC/cell and percentage of HBBAS3 mRNA and correlation of VC/cell and percentage of HbAS3; Spearman correlation (Lehmann and D'Abrera (2006) Nonparametrics: Statistical Methods Based on Ranks. New York, N.Y., USA: Springer) was used to evaluate the correlation of the VC/cell with the percentage of corrected sickle cell. For binary outcome, such as the replating condition in the IVIM assay (positive vs. negative), Fisher's exact test (Fisher (1922) J. R. Stat. Soc. 85(1): 87-94) was used to compare CCL-βAS3-FB vector with RSF91-GFP vector. To compare the proportions of IS near cancer-related genes in cells grown in vitro with cells engrafted in mice, a binomial test was performed using the proportion of cancer gene-proximal IS in the in vitro condition as an estimate of the probability of observing such an IS in engrafted mice. For all statistical investigations, tests for significance were 2 tailed. P<0.05 was considered to be statistically significant. All statistical analyses were carried out using SAS version 9.3 (SAS Institute (2011). SAS/STAT 9.3 User's Guide: The REG Procedure (Chapter). Carey, N.C., USA: SAS Institute, Inc.), Graph-Pad Prism version 5.0d (GraphPad Software Inc.), and MATLAB version 7.12.0.635 (MathWorks Inc.).

Study Approval.

All human samples have been used following UCLA IRB protocol #10-001399. Written informed consent was obtained from the subjects used in these studies. All work with mice was done under protocols approved by the UCLA Animal Care Committee.

### Supplemental Materials and Methods.

CCL-βAS3-FB LV Vector Construction

The HBBAS3 cassette (human HBB gene with 3 amino acid substitutions, HBB promoter, 3′ HBB enhancer, and DNAase hyper-sensitive sites HS2, HS3 and HS4) and the WPRE were amplified by PCR from the DL-βAS3 LV plasmid (Levasseur et al. (2003) Blood, 102(13): 4312-4319) (generously provided Tim Townes, UAB, Birmingham, Ala.) using AccuPrime Pfx DNA polymerase (Invitrogen, Carlsbad, Calif.) with the primers AS3-forward (F)- and AS3-reverse (R)-. The 6.6 Kb PCR product was purified by PureLink QuickGel Extraction Kit (Invitrogen, Carlsbad, Calif.) and subcloned into the plasmid pCR2.1-TOPO-TA (Invitrogen, Carlsbad, Calif.). To include the FB insulator in the 3′LTR of the pCCLcPPT-x-plasmid, a PCR reaction was done using pHR′-CMV-EGFP to generate a 1-LTR (SIN) plasmid, using the primers: PHR′3′LTR-amp-ori F and PHR′ 3′LTR-amp-ori R2. The 1-LTR plasmid was digested with EcoRV and PvuII, phosphatase treated and ligated with a phosphorylated oligonucleotide cassette containing the FB (77 bp) insulator sequence (CCC AGG GAT GTA CGT CCC TAA CCC GCT AGG GGG CAG CAC CCA GGC CTG CAC TGC CGC CT GCC GGC AGG GGT CCA GTC (SEQ ID NO:6)) (Ramezani et al. (2008) Stem Cells, 26(12): 3257-3266) to obtain the 1-LTR-FB plasmid.

After verifying the 1-LTR-FB clone, PCR was performed with the 1-LTR-FB plasmid with primers 3′LTR F (Vostrov and Quitschke (1997) J. Biol. Chem. 272(52): 33353-33359) and 3′LTR R (Id.); and then with the pCCL-cPPT empty backbone using the primers pCCL LTR insert F (Wu et al. (2003) Science 300(5626): 1749-1751) and pCCL LTR insert R (Brunak et al. (1991) J. Mol. Biol. 220(1): 49-65). These PCR products were used in an In-Fusion reaction (Clontech Laboratories, Inc, Mountain View Calif.). The two fragments overlapped at the 3′ LTR, making the pCCL-cPPT-x-FB backbone. The pCCL-cPPT-x-cHS4 backbone was created by digesting the 1-LTR plasmid created from pHR′, as described above, with EcoRV and PvuII. The 1.2 kb cHS4 insulator was amplified using primers 1.2 kb-F and 1.2 kb-R. The resulting product was cloned into the linearized 1-LTR plasmid via In-Fusion (Clontech Laboratories, Inc, Mountain View Calif.). The full 3′ LTR was transferred to pCCL-cPPT-x as described above for the FB-containing LTR. To include the PAS3-WPRE fragment into the pCCL-cPPT-x-backbone, the PCR2.1-TOPO-PAS3-WPRE plasmid was digested with Seal and Kpnl, the purified product was blunted and digested with Xhol. The 6.6 kb band corresponding to the PAS3-WPRE fragment was isolated by gel purification and cloned into the pCCL-cPP-x-backbone, previously digested by EcoRV and Xhol. The resulting pCCL-cPPT-PAS3-WPRE (called CCL-PAS3) vector plasmid was fully sequenced to verify the construction. The same procedure was performed to develop the insulated versions CCL-PAS3-FB and CCL-PAS3-cHS4, cloning the PAS3-WPRE cassette in the previously described pCCL-cPPT-x-FB and pCCL-cPPT-x-cHS4 backbones, respectively. (Primers sequences are provided in Table 6).

TABLE 6
Oligonucleotide sequences.

| | | SEQ |
|---|---|---|
| Primer Name | Sequence (5′-3′) | ID NO |
| AS3-F | CTACTAGTGGAGATCCC | 7 |
| AS3-R | GAAGCTTGAGCGAATTC | 8 |
| PHR′ 3′LTR-amp-ori F | GGGACTGGAAGGGCTAATTCACTC | 9 |
| PHR′ 3′LTR-amp-ori R2 | CCAGCAAAAGGCCAGGAACC | 10 |
| 3′LTR F (58) | GGGACTGGAAGGGCTAATTC | 11 |
| 3′LTR R (58) | CCTCTCACTCTCTGATATTCATTTCTT | 12 |
| pCCL LTR insert F (60) | AGCCCTTCCAGTCCCCC | 13 |
| pCCL LTR insert R (59) | TCAGAGAGTGAGAGGAACTTGTTTATT | 14 |
| 5′LTR-F | GGCTAATTCACTCCCAACGAAGACAAG | 15 |
| 5′LTR-R | CTT CAG CAA GCC GAG TCC TGC | 16 |
| 3′LTR-F | ACC TCG AGA CCT AGA AAA ACA TGG C | 17 |
| 3′LTR-R | CAGAGAGACCCAGTACAAGCAAAAAG | 18 |
| HBB^{AS3} F | TGTGGGACAAGGTGAACGTGGATGCC | 19 |
| HBB^{AS3} R | CAAGGGTAGACCACCAGCAGCCTG | 20 |
| HBB^{A}/HBB^{S} F | TGTGGGGCAAGGTGAACGTGGATGAA | 21 |
| HBB^{A}/HBB^{S} R | CAAGGGTAGACCACCAGCAGCCTG | 22 |
| FB-F | ACTCCCAACGAAGACAAGATCCCA | 23 |
| FB-R | ACCAGAGAGACCCAGTACAAGCAA | 24 |
| cHS4-F | GTAATTACGTCCCTCCCCCG | 25 |
| cHS4-R | AAGCGTTCAGAGGAAAGCGA | 26 |
| U3-F | ACTCCCAACGAAGACAAGATCTGC | 27 |
| U3-R | ATTGAGGCTTAAGCAGTGGGTTCC | 28 |
| H19-F | AGAATCGGCTGTACGTGTGG | 29 |
| H19-R | GGGACGTTTCTGTGGGTGAA | 30 |
| Myc-F | GCCATTACCGGTTCTCCATA | 31 |
| Myc-R | CAGGCGGTTCCTTAAAACAA | 32 |
| ddHBB^{AS3}-F | GGAGAAGTCTGCCGTTACTG | 33 |
| ddHBB^{AS3}-R | CACTAAAGGCACCGAGCACT | 34 |
| ddHBB^{AS3}Probe | FAM-ACAAGGTGA-ZEN- | 35 |
| | ACGTGGATGCCGTTG-31ABFQ | |

Production and Titration of pAS3-Globin LV

For small-scale production of LV for titer analysis, 293T cells (5×10⁶) (ATCC, Manassas, Va.) were seeded per 10 cm cell culture dishes coated with Poly L-Lysine (Sigma-Aldrich, St. Louis, Mo.) in 10 ml of D10 medium, consisting of DMEM (Mediatech, Herndon, Va.) with 10% fetal bovine serum (FBS) (Gemini Bio-products, Sacramento, Calif.), 1× Glutamine, Penicillin and Streptomycin (Gemini Bio-Products, West Sacramento, Calif.), 24 hours before transfection. On the day of the transfection, 3 pl of TransIT-293 (Mirus, Madison, Wis.) were used per 1 pg of DNA. The TransIT volume needed for each condition was mixed with 500 ul of OPTI-MEM (Invitrogen, Carlsbad, Calif.), vortexed and incubated for 20 minutes at room temperature. The OPTI-MEM/TransIT solution was mixed with (a) 5 pg of the transfer plasmid, (b) 5 pg of pMDL gag-pol/pRRE, (c) 2.5 pg of pRSV-Rev (both were kind gifts of Luigi Naldini, CellGenesys, Foster City, Calif.), and (d) 1 pg of pMDG-VSV-G (3). In the transfections that were done with TAT, 2.5 pg of pSV2-tat were used (4) (provided by the NIH AIDS Research and Reagent Program, Germantown, Md.). The DNA and OPTI-MEM/TransIT solutions were incubated for 15-30 minutes at room temperature. The 293T cells were washed with 10 ml of D10 before adding the transfection mixture to each plate. Approximately 18-20 hours post-transfection, the medium on the transfected cells was changed to medium containing 10 mM sodium butyrate (Sigma-Aldrich, St. Louis, Mo.) and 20 mM HEPES (Invitrogen, Carlsbad, Calif.). After 6-8 hours, the cells were washed with DPBS (Mediatech, Herndon, Va.) and 6 ml of medium containing 20 mM HEPES were added. After 48 hours, the vectors were harvested, filtered (0.45 pm) and titered by qPCR as described previously (Cooper et al. (2011) J. Virol. Meth. 177(1): 1-9). Large-scale viral preparations were produced and concentrated using tangential flow filtration and titered by qPCR as described previously (Id.).

FB Insulator Integrity in the CCL-PAS3-FB Provirus

The integrity of the FB insulator was analyzed by PCR from both LTRs in transduced BM CD34⁺ cells at day 14 of in vitro erythroid culture after genomic DNA isolation using the PureLink Genomic DNA Mini Kit (Invitrogen, Carlsbad, Calif.). A first set of primers was designed (5′LTR-F and 5′LTR-R) to amplify the 5′ LTR flanking the FB insertion site, with an expected band of 382 bp when the FB insulator was present and intact. The second set of primers was designed (3′LTR-F and 3′LTR-R) to amplify specifically the 3′ LTR; in this case the predicted band was 249 bp in the presence of the FB insulator. A third PCR reaction was performed combining the 5′ LTR-F and the 3′ LTR-R to amplify the FB insulator by itself from both LTRs. In this case the corresponding amplicon had a length of 135 bp. (All primers sequence provided in Table 6). PCR was executed using Taq DNA Polymerase, Native (Invitrogen, Carlsbad, Calif.) on an Eppendorf (Hamburg, Germany) thermocycler. PCR products were visualized by GelGreen on 2% agarose gels.

Southern Blot

Southern blot analysis was performed to confirm the integrity of the CCL-PAS3-FB LV provirus in the genome. 293T cells were transduced with the CCL-PAS3-FB LV and expanded over two weeks, followed by genomic DNA isolation (Invitrogen, Carlsbad, Calif.). 10 pg of genomic DNA was digested by Afl II (New England Biolabs, Ipswich, Mass.), electrophoresed at 20 volts overnight in a 0.8% agarose gel, transferred to a nylon membrane and probed with a ³²P-labelled-WPRE fragment overnight.

Chromatin Immunoprecipitation (ChIP)

K562 cells (ATCC # CCL-243™) were transduced with CCL-pAS3, CCL-pAS3-FB and CCL-pAS3-cHS4 LV vectors at a concentration of 2×10⁸ TU/ml for each vector. 2×10⁷ transduced K562 cells were collected, washed with PBS and cross-linked by incubation in 1% formaldehyde for 5 minute at room temperature. Nuclei were isolated using the truChIP Low Cell Chromatin Shearing Kit (Covaris, Woburn, Mass.), and the DNA-protein complexes were sheared for 6 minutes in a COVARIS M220 sonicator per manufacturer instructions. Sheared chromatin was immuno-precipitated (in triplicate) for 12-16 h at 4° C. using 5 pg of anti-CTCF antibody (Abcam, Cambridge, Mass.) or rabbit IgG as negative control (Invitrogen, Carlsbad, Calif.) following the “MAGnify Chromatin Immunoprecipitation System” protocol (Invitrogen, Carlsbad, Calif.). After reversing the cross-linking, DNA was quantified using “Quant-IT PicoGreen dsDNA Reagent and Kits” (Molecular Probes, Invitrogen, Carlsbad, Calif.). The same amounts of DNA from CTCF immuno-precipitated, IgG control and input DNA samples were used to perform real-time qPCR in triplicate using the Viia7 Applied Biosystems real time PCR machine with the following conditions: hold stage: 50° C. for 2 minutes, 95° C. for 10 minutes; PCR stage: 95° C. for 15 seconds, 60° C. for 1 minute (40 cycles). (Primers sequences are described in Table 6). Data were analyzed using relative quantitation method as described in the ABI User Bulletin #2 “Relative quantitation of gene expression” (Biosystems A. ABI PRISM 7700 Sequence Detection System. ABI PRISM 7700 Sequence Detection System. 2001; (User Bulletin #2):1-36), and Litt et al. (2001) EMBO J. 20(9): 2224-2235. In brief, fold enrichment for a particular target sequence was determined using the following formula: fold enrichment=AE(Ct input-ct IP). AE=amplification efficiency, input=amount of the target sequence in input DNA; IP=amount of target sequence in immune-precipitated DNA.

BM CD34+ Cell Isolation

Human CD34⁺ cells were isolated from BM aspirates from HD and SCD donors (beta^{S}/beta^{S} or beta^{S}/betathal⁰). The mononuclear fractions obtained by density gradient centrifugation on Ficoll-Hypaque (Amersham Pharmacia Biotech Piscataway, N.J.) were processed using the Human CD34 Microbead kit (Miltenyi Biotech, Bergisch Gladbach, Germany) and the CD34⁺ cells recovered were cryopreserved.

CFU Progenitor Assay in Methylcellulose

100, 300 and 1000 BM CD34⁺ cells (non-transduced or transduced) were plated per 35 mm gridded cell culture dish in duplicate, using methylcellulose medium (Stem Cell Technologies, Vancouver, BC, Canada) enriched to support optimal growth of human hematopoietic progenitors from CD34⁺-enriched cells. After 14 days of culture at 5% CO₂, 37° C. and humidified atmosphere, the different types of colonies were identified based in their morphology, and then counted and plucked for genomic DNA isolation (NucleoSpin Tissue XS, Clontech, Mountain View, Calif.) for determination of VC/cell by qPCR as described before (Cooper et al. (2011) J. Viral. Meth. 177(1): 1-9).

Myeloid Culture

In parallel to the erythroid culture 5×10⁴ cells per condition were grown in myeloid conditions for 14 days to measure VC/cell by qPCR as described before (Id.). The basic myeloid medium consists of IMDM supplemented with 20% of FBS (Life Technologies, Grand Island, N.Y.), 35% BSA (Sigma-Aldrich, St. Louis, Mo.), 1× Glutamine, Penicillin and Streptomycin, 5 ng/ml hIL-3, 10 ng/ml hIL-6 (both from R&D) and 25 ng/ml hSCF (StemGent, Cambridge, Mass.).

Flow Cytometry During Erythroid Culture

At days 3, 14 ad 21 of the in vitro erythroid differentiation, 2×10⁵ cells were collected for flow cytometry analysis. The samples were stained with the following antibodies: phycoerythrin (PE)-conjugated anti-human CD34, V450-conjugated anti-human CD45, allophycocyanin (APC)-conjugated anti-human CD71 (all from BD Biosciences, San Jose, Calif.) and fluorescein isothyocyanate (FITC)-conjugated anti-GlycophorinA (GpA) (Santa Cruz Biotechnologies, Santa Cruz, Calif.). At day 21, the percentage of enucleated RBC produced was measured by double staining: DRAQ5 (Biostatus Limited, UK) for nuclear staining and FITC-conjugated anti-GpA; enucleated RBC were defined as being GpA+/DRAQ5−. All the flow cytometry analyses were performed on an LSR Fortessa cell analyzer (BD Biosciences, San Jose, Calif.).

In Vitro Immortalization (IVIM) Assay

To obtain lineage-negative (stem cell enriched) populations from BM, untreated 7- to 12-week-old male B6.SJL-PtprcaPepcb/BoyJ (“Pep Boys”) were used as donors. BM cells were collected from the long bones (2 femurs, 2 tibias and 2 humeri) of each mouse into IMDM supplemented with 10% FBS. Lineage-negative cells were isolated from single cell suspensions of whole BM cells by using the Lineage Cell Depletion Kit (Miltenyi Biotec, Bergisch Gladbach, Germany) according to manufacturer's instructions and cryopreserved in aliquots. Upon thawing, lineage-negative cells were pre-stimulated in StemSpan SFEM serum-free expansion medium (STEMCELL Technologies Inc., Vancouver, Canada) containing 50 ng/ml mSCF, 10 ng/ml human Interleukin-11 (hIL-11), 20 ng/ml mIL-3 (all PeproTech Inc., Rocky Hill, N.J., USA), 100 ng/ml hFlt3-L (Celldex Therapeutics, Needham, Mass.) and 1× Glutamine, Penicillin and Streptomycin in Retronectin (20 p, g/ml) coated wells of 24 well plates at a concentration of 0.5-1×10⁶ cells/ml for 2 days before exposure to vector particles. For retroviral transduction, RSF91-GFP-WPRE viral particles were preloaded onto Retronectin coated wells of 24 well plates by centrifugation at 1000 g for 30 minutes at 40° C. at multiplicity of infection ranging from 1 to 20. The viral supernatant was aspirated, and 1×10⁵ pre-stimulated lineage negative cells were added in 500 μL StemSpan medium containing cytokines on day 3. On day 4, cells were transferred to a new 24 well plate, freshly preloaded with retroviral particles in 1 mL to account for increasing cell numbers. For LV transduction, 110⁵ pre-stimulated lineage-negative cells were transduced with concentrated CCL-βAS3, CCL-βAS3-FB and CCL-βAS3-cHS4 LV supernatants at 2×10⁷ TU/mL and 2×10⁸ TU/ml in 500 μL StemSpan medium containing cytokines on day 3. On day 4, 500 μL medium was added to account for increasing cell numbers. Starting on day 5 (day 1 pTD), mock-, retroviral-, and lentiviral-transduced samples were expanded as mass cultures for 2 weeks in IMDM supplemented with 10% FBS, 1× Glutamine, Penicillin and Streptomycin, 50 ng/ml mSCF, 100 ng/ml hIL-11, 20 ng/ml mIL-3 and 100 ng/ml hFlt3-L. During this time, cell density was adjusted to 5×10⁵/ml on days 4, 6, 8, 11, and 13 pTD. On day 15 pTD, cells were plated in a limiting dilution assay in 96 well plates at a density of 100 cells/well and 1000 cells/well, respectively, in 100 μl IMDM supplemented with FBS, Glutamine, Penicillin, Streptomycin and cytokines Two weeks later the positive wells were counted, and the frequency of replating cells was calculated based on Poisson statistics using L-Calc Software (STEMCELL Technologies Inc., Vancouver, Canada).

HBBAS3 mRNA Expression in Erythroid and Myeloid Conditions

After BM-CD34⁺ cells transduction, samples were divided into parallel cultures under myeloid and erythroid differentiation conditions. At 14 day of culture, 1.5×10⁵ cells were harvested for each group. RNA extraction and cDNA synthesis were performed as described in the Materials and Methods section. The ddHBBAS3 assay sequences are provided in Table 6. The P-Actin, ACTB (Hs 99999903_m1), was purchased as a 20×-premix of primers and FAM-MGBNFQ probe (Applied Biosystems, San Francisco, Calif.). Reaction mixtures of 20 μl volume comprising 1×ddPCR Master Mix (Bio-Rad, Hercules, Calif.), relevant primers and probe (900 nM and 250 nM for ACTB primers and probe respectively; 500 nM and 100 nM for ddHBB^{AS3} primers and probe), and 1 μl of cDNA were prepared. Droplet generation was performed as described in Hindson et al. (2011) Anal. Chem. 83(22): 8604-8610. The droplet emulsion was then transferred with a multichannel pipet to a 96-well propylene plate (Eppendorf, Hamburg, Germany), heat sealed with foil, and amplified in a conventional thermal cycler (T100 Thermal Cycler, Bio-Rad). Thermal cycling conditions consisted of 95° C. 10 min, 94° C. 30 s and 60° C. 1 min (55 cycles), 98° C. 10 min (1 cycle), and 12° C. hold. After PCR, the 96-well plate was transferred to a droplet reader (Bio-Rad). Acquisition and analysis of the ddPCR data was performed with the QuantaSoft software (Bio-Rad), provided with the droplet reader. The relative expression of HBBAS3/ACTB was calculated by dividing the concentration (copies/μ1) of HBBAS3 by the concentration of ACTB, and normalized to the VC/cell.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A recombinant lentiviral vector (LV) comprising:
an expression cassette comprising a nucleic acid construct comprising an anti-sickling human beta globin gene encoding an anti-sickling-beta globin polypeptide comprising the mutations Gly16Asp, Glu22Ala and Thr87Gln;
where said LV is a TAT-independent and self-inactivating (SIN) lentiviral vector.

2. The vector of claim 1, wherein said anti-sickling human β-globin gene comprises about 2.3 kb of recombinant human β-globin gene including exons and introns under the control of the human β-globin gene 5′ promoter and the human β-globin 3′ enhancer.

3. The vector claim 2, wherein said β-globin gene comprises β-globin intron 2 with a 375 bp RsaI deletion from IVS2, and a composite human β-globin locus control region comprising HS2, HS3, and HS4.

4. The vector of claim 1, further comprising an insulator in the 3′ LTR.

5. The vector of claim 4, wherein said insulator comprises FB (FII/BEAD-A), a 77 bp insulator element, which contains the minimal CTCF binding site enhancer-blocking components of the chicken β-globin 5′ DnaseI-hypersensitive site 4 (5′ HS4).

6. The vector of claim 1, wherein said vector comprises a ψ region vector genome packaging signal.

7. The vector of claim 1, wherein the 5′ LTR comprises a CMV enhancer/promoter.

8. The vector of claim 1, wherein said vector comprises a Rev Responsive Element (RRE).

9. The vector of claim 1, wherein said vector comprises a central polypurine tract.

10. The vector of claim 1, wherein said vector comprises a post-translational regulatory element.

11. The vector of claim 10, wherein the posttranscriptional regulatory element is modified Woodchuck Post-transcriptional Regulatory Element (WPRE).

12. The vector of claim 1, wherein said vector is incapable of reconstituting a wild-type lentivirus through recombination

13. A host cell transduced with a vector of claim 1.

14. The host cell of claim 13, wherein the cell is a stem cell.

15. The host cell of claim 14, wherein said cell is a stem cell derived from bone marrow.

16. The host cell of claim 13, wherein the cell is a 293T cell.

17. The host cell of claim 13, wherein, wherein the cell is a human hematopoietic progenitor cell.

18. The host cell of claim 17, wherein the human hematopoietic progenitor cell is a CD34⁺ cell.

19. A method of treating sickle cell disease in a subject, said method comprising:
transducing a stem cell and/or progenitor cell from said subject with a vector of claim 1;
transplanting said transduced cell or cells derived therefrom into said subject where said cells or derivatives therefrom express said anti-sickling human beta globin gene.

20. The method of claim 19, wherein the cell is a stem cell.

21. The host cell of claim 19, wherein said cell is a stem cell derived from bone marrow.

22. The method of claim 19, wherein, wherein the cell is a human hematopoietic progenitor cell.

23. The method of claim 22, wherein the human hematopoietic progenitor cell is a CD34⁺ cell.
